(19) United States
(12) Patent Application Publication
(10) Pub. No.: US 2007/0025987 A1
(43) Pub. Date: Feb. 1, 2007

(54) Method for Treating Vasculitis

(75) Inventor: Paul G. Brunetta, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/536,571

(22) Filed: Sep. 28, 2006

Related U.S. Application Data
(63) Continuation of application No. 11/238,281, filed on Sep. 28, 2005.
(60) Provisional application No. 60/616,104, filed on Oct. 5, 2004.

(51) Int. Cl. A61K 39/395; A61K 31/66; A61K 31/573; A61K 31/5377; A61K 31/525; A61K 31/519

(52) U.S. Cl. 424/144.1; 514/171; 514/232.8; 514/262.1; 514/251; 514/109

(57) ABSTRACT

A method of treating anti-neutrophil cytoplasmic antibodies-associated vasculitis (ANCA-associated vasculitis) in a patient eligible for treatment is provided involving administering an antagonist that binds to a B-cell surface marker, such as CD20 antibody, to the patient in a dose of about 400 mg to 1.3 grams at a frequency of one to three doses within a period of about one month. Another method of treating ANCA-associated vasculitis in a subject eligible for treatment is provided involving administering an effective amount of an antibody that binds to a B-cell surface marker to the subject to provide an initial exposure and a subsequent exposure to the antibody within certain dosing regimens. Further provided are articles of manufacture useful for such methods.

### RELATED APPLICATIONS

This application is a continuation application of Ser. No. 11/238,281 filed on Sep. 28, 2005, which application claims priority to provisional application number 60\616,104 filed Oct. 5, 2004, the contents of which are incorporated herein by reference.

### FIELD OF THE INVENTION

The present invention concerns methods for treating anti-neutrophil cytoplasmic antibodies (ANCA)-associated vasculitis in a subject, and kits with instructions for such uses.

### BACKGROUND OF THE INVENTION

### Vasculitis

Autoimmune diseases, such as rheumatoid arthritis, multiple sclerosis, vasculitis, and lupus, among others, remain clinically important diseases in humans. As the name implies, autoimmune diseases wreak their havoc through the body's own immune system. While the pathological mechanisms differ among individual types of autoimmune diseases, one general mechanism involves the binding of certain antibodies (referred to herein as self-reactive antibodies or autoantibodies) present.

Vasculitis is defined by inflammation of the blood-vessel wall and forms the pathological foundation of a diverse group of individual disease entities. Anti-neutrophil cytoplasmic antibodies (ANCA)-associated vasculitis, which is a common primary systemic vasculitis, includes microscopic polyangiitis, Wegener's granulomatosis, Churg-Strauss syndrome, renal-limited vasculitis (idiopathic necrotizing crescentic glomerulonephritis) (Falk et al. N. Engl. J. Med., 318: 1651-1657 (1988)), and certain types of drug-induced vasculitis. Jennette et al. Arthritis Rheum 37:187-92 (1994); Jennette and Falk, N. Engl. J. Med. 337:1512-1523 (1997). The diseases mentioned above affect people of all ages but are most common in older adults in their 50s and 60s, and they affect men and women equally. Pettersson et al, Clin. Nephrol., 43: 141-149 (1995); Falk et al., Ann. Intern. Med. 113: 656-663 (1990).

ANCA are specific antibodies for antigens in cytoplasmic granules of neutrophils and monocyte lysosomes, first reported in 1982. Niles et al., Arch. Intern. Med., 156:440-445 (1996). ANCA were originally detected by indirect immunofluorescence on ethanol-fixed neutrophils. Wiik, “Delineation of a standard procedure for indirect immunofluorescence detection of ANCA” APMIS Suppl. 6: 12-13 (1989). At least three different patterns of fluorescence have been distinguished: a cytoplasmic/classic pattern (cANCA) with accentuation of the fluorescence intensity in the area with the nuclear lobes, a perinuclear pattern (pANCA), and a more diffuse cytoplasmic staining pattern (atypical ANCA). Approximately 90% of the sera that produce a cANCA pattern react with proteinase 3 (PR3), a serine protease from the azurophilic granules of myeloid cells. Jennette and Falk, N Engl. J. Med., supra. In patients with primary systemic vasculitis predominantly affecting medium- and small-sized blood vessels, approximately 75% of the sera producing a perinuclear pattern (pANCA) react with myeloperoxidase (MPO), a myeloid lysosomal enzyme. Cohen Tervaert et al., Am. J. Med. 91: 59-66 (1991). In ANCA-positive patients with other non-vasculitic diseases, often antigenic specifiides are recognized. The diagnostic potential of PR3-ANCA and MPO-ANCA is now fairly well established. In a patient with signs and symptoms of vasculitis, ANCA with specificity for PR3 (PR3-ANCA) suggests a diagnosis of Wegener's granulomatosis, whereas ANCA with a specificity for MPO (MPO-ANCA) is highly sensitive for microscopic polyangiitis, idiopathic necrotizing crescentic glomerulonephritis, or active Churg-Strauss syndrome. Cohen Tervaert et al., Sarcoidosis Vasc. Diffuse Lung Dis. 13: 241-245 (1996). See also Xiao et al., J. Clin. Invest., 110: 955-963 (2002), which describe an animal model that offers strong support for a direct pathogenic role for ANCA IgG in human glomerulonephritis and vasculitis, and Popa et al., J. Allergy Clin. Immunol., 103: 885-894 (1999) showing that in Wegener's granulomatosis, B-cell activation is related to active disease, whereas T-cell activation persists during remission of the disease, which points to an intrinsic disordered immune system in this disease. See also Cupps et al., J. Immunol. 128: 2453-2457 (1982) regarding the role of cyclophosphamide in suppressing human B lymphocyte function.

Within the spectrum of primary vasculitic syndromes, the ANCA-related syndromes form a distinct group with overlapping features. Most patients have a prodromal flu-like onset consisting of malaise, myalgias, arthralgias, fever, and weight loss. This flu-like onset appears within days to weeks before the onset of overt vasculitic or nephritic disease. Wegener's granulomatosis is differentiated from the others by the presence of necrotizing granulomatous inflammation of the upper and lower respiratory tract, which is usually accompanied by systemic necrotizing small vessel vasculitis and glomerulonephritis. Churg-Strauss syndrome is differentiated by the presence of (a history of) asthma, allergic rhinitis, systemic eosinophilia, in addition to systemic vasculitis with or without glomerulonephritis. Microscopic polyangiitis is characterized by necrotizing and/or crescentic glomerulonephritis and a multi-system vasculitis involving small vessels. Microscopic polyangiitis shares many features with Wegener's granulomatosis and Churg-Strauss syndrome, but lacks necrotizing granulomatous inflammation of the respiratory tract and asthma. Jennette et al., Arthritis Rheum., supra. In idiopathic necrotizing and/or crescentic glomerulonephritis the vasculitic process is limited to the kidneys. Because the treatment of patients with microscopic polyangiitis or Wegener's granulomatosis is essentially the same when there is major organ injury, it is unnecessary to distinguish conclusively between these closely related variants of ANCA-associated vasculitis before initiating treatment. Jennette et al. Arthritis Rheum., supra.

Before treatment became available, patients with generalized Wegener's granulomatosis had a median survival of five months. In the early 1970s, Fauci and Wolff introduced a regimen combining daily cyclophosphamide therapy given for one year after remission was achieved with prednisone therapy initiated at a dose of 1 mg per kilogram of body weight per day and tapered on an alternate-day schedule. This treatment has reproducibly been found to induce remission in 80 to 100 percent of patients and can result in long-term survival. In fact, prolonged immunosuppressive therapy (greater than 1 year) with cyclophosphamide and steroids is effective in inducing disease remission and preventing early relapses in most vasculitic disorders. Balow et al., “Vasculitic diseases of the kidney, polyarteritis, Wegener's granulomatosis, necrotizing and crescentic glomerulonephritis, and other disorders.” In: Schrier and Gottschalk (eds): Diseases of the kidney, 5^{th} edition, (Little, Brown and Company, Boston, 1993), pp. 2095-2117; Jayne et al., N. Engl. J. Med., 349: 36-44 (2003); Gaskin et al, “Systemic vasculitis” In: Cameon et al. (eds): Oxford textbook of clinical nephrology. (Oxford University Press, Oxford, 1992), pp. 612-636; Fauci et al, Ann. Intern. Med. 89: 660-676 (1978); Fauci et al., Ann. Intern. Med., 98: 76-85 (1983); Hoffman et al., Ann. Int. Med., 116: 488-498 (1992); and Andrassy et al., Clin. Nephrol., 35: 139-147 (1991).

However, when therapy is tapered and discontinued, relapses are common. In one study, in which patients with Wegener's granulomatosis were followed for a mean of eight years, relapse occurred in 50 percent of patients. Further, continuous use of cyclophosphamide to sustain remission is not recommended, since this treatment regimen is associated with severe and potentially lethal adverse effects such as the occurrence of opportunistic infections and the development of malignancies. For example, repeated courses of cyclophosphamide are associated with bone-marrow suppression, infection, cystitis, infertility, myelodysplasia, and transitional-cell carcinoma of the bladder. In some instances, such toxic effects preclude further use of cyclophosphamide. Stillwell et al., Arthritis Rheum., 31: 465-470 (1988); Radis et al., Arthritis Rheum. 38: 1120-1127 (1995). Therefore, cyclophosphamide is tapered or stopped and replaced by azathioprine once remission is achieved to prevent adverse effects, a policy tested in a rigorous multi-center trial and proven to be equally effective in the follow-up for 18 months. Gaskin et al, supra, 1992; Jayne, Rheumatology 39: 585-595 (2000). Azathioprine is considered less effective in inducing remission than cyclophosphamide, but its long-term toxicity is much lower. Bouroncle et al., Am. J. Med., 42: 314-318 (1967); Norton et al., Arch. Intern. Med., 121: 554-560 (1968).

Other alternative maintenance therapy regimens include methotrexate ((de Groot et al, Arthritis Rheum., 39: 2052-2061 (1996)), cyclosporine A (Haubitz et al., Nephrol. Dial. Transplant, 13: 2074-2076 (1998)), mycophenolate (Nowack et al., J. Am. Soc. Nephrol., 10: 1965-1971 (1999)), or trimethoprim-sulfamethoxazole (Stegeman et al., N. Engl. J. Med., 335: 16-20 (1996)). See also Sanders, et al. N. Engl. J. Med. 349: 2072-2073 (2003). Since, however, relapses are frequently observed in ANCA-associated vasculitis, treatment in such cases has to be intensified or reinstituted. Hoffman et al, supra; Gordon et al, Q J. Med., 86: 779-789 (1993); Nachman et al., J. Am. Soc. Nephrol., 7: 33-39 (1996); Guillevin et al., Medicine 78: 26-37 (1999); Reinhold-Keller et al., Arthritis. Rheum. 43: 1021-1032 (2000); Langford, New Eng. J. Med., 349: 3-4 (July 2003).

Tumor necrosis factor-alpha (TNF-alpha) blockade with infliximab is a potential therapy for ANCA-associated vasculitis, both for initial therapy and in the management of refractory disease. Infliximab was effective at inducing remission in 88% of patients with ANCA-associated vasculitis and permitted reduction in steroid doses. Booth et al., J. Am. Soc. Nephrol. 15:717-721 (2004). In addition, Stone et al., Arthritis and Rheumatism, 44: 1149-1154 (2001) disclosed that the TNF-alpha inhibitor etanercept (ENBREL®), given 25 mg subcutaneously twice weekly in combination with standard treatment for Wegener's granulomatosis, was well-tolerated in the patients with few adverse events, but intermittently active disease (occasionally severe) was common.

Patients with Churg-Strauss syndrome usually respond to high-dose corticosteroid therapy alone, although some cases may require the addition of cytotoxic drugs. Jayne and Rasmussen, Mayo Clin. Proc. 72:737-47 (1997). Co-morbid conditions that accelerate vascular damage, e.g., hypertension, diabetes, hypercholesterolemia, and smoking, should be appropriately controlled.

In drug-induced vasculitis, the offending agent should be stopped. Antihistamines and non-steroidal anti-inflammatory drugs help alleviate skin discomfort and reduce associated arthralgias and myalgias. Severe cutaneous disease may warrant oral corticosteroid therapy. Jennette et al., Arthritis Rheum, supra.

The persistence or reappearance of ANCA is a risk factor for the development of a relapse of disease activity, suggesting a pathophysiological role in vivo for these autoantibodies. Stegeman et al., Ann. Intern. Med., 120: 12-17 (1994); De'Oliviera et al, Am. J. Kidney Dis., 25: 380-389 (1995); Jayne et al., Q. J. Med., 88: 127-133 (1995). Relapses of Wegener's granulomatosis are frequently preceded by rises in the titer of cANCA as detected by indirect immunofluorescence (Cohen Tervaert et al., Arch. Intern. Med., 149: 2461-2465 (1989)), and can be prevented by treatment with immunosuppressives based on rises in cANCA (Cohen Tervaert et al., Lancet, 336: 706-711 (1990)).

For a general discussion on ANCA-associated vasculitis, see Lhote and Guillevin, Rheum. Dis. Clin. North Am. 21:911-947 (1995); “ANCA-associated vasculitis: occurrence, prediction, prevention, and outcome of relapses” by Maarten Boomsma, PhD Thesis, Thesis University Groningen, ISBN 90-367-1451-6 (M. M. Boomsma, Groningen, 2001) (http://www.ub.rug.nl/eldoc/dis/medicine/m.m.boomsmalthesis.pdf); Kamesh et al., J. Am. Soc. Nephrol. 13:1953-1960 (2002); and Jayne, Kidney & Blood Pressure Research 26:231-239 (2003).

### CD20 Antibodies and Therapy Therewith

Lymphocytes are one of many types of white blood cells produced in the bone marrow during the process of hematopoiesis. There are two major populations of lymphocytes: B lymphocytes (B cells) and T lymphocytes (T cells). The lymphocytes of particular interest herein are B cells.

B cells mature within the bone marrow and leave the marrow expressing an antigen-binding antibody on their cell surface. When a naïve B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody, but instead produce the antibody in a form that can be secreted. Secreted antibodies are the major effector molecules of humoral immunity.

The CD20 antigen (also called human B-lymphocyte-restricted differentiation antigen, Bp35) is a hydrophobic transmembrane protein with a molecular weight of approximately 35 kD located on pre-B and mature B lymphocytes. Valentine et al., J. Biol. Chem. 264(19):11282-11287 (1989) and Einfeld et al., EMBO J. 7(3):711-717 (1988). The antigen is also expressed on greater than 90% of B-cell non-Hodgkin's lymphomas (NHL) (Anderson et al. Blood 63(6):1424-1433 (1984)), but is not found on hematopoietic stem cells, pro-B cells, normal plasma cells, or other normal tissues (Tedder et al. J. Immunol. 135(2):973-979 (1985)). CD20 regulates an early step(s) in the activation process for cell-cycle initiation and differentiation (Tedder et al., supra), and possibly functions as a calcium-ion channel. Tedder et al., J. Cell. Biochem. 14D: 195 (1990).

Given the expression of CD20 in B-cell lymphomas, this antigen can serve as a candidate for “targeting” of such lymphomas. In essence, such targeting can be generalized as follows: antibodies specific to the CD20 surface antigen of B cells are administered to a patient. These anti-CD20 antibodies specifically bind to the CD20 antigen of (ostensibly) both normal and malignant B cells; the antibody bound to the CD20 surface antigen may lead to the destruction and depletion of neoplastic B cells. Additionally, chemical agents or radioactive labels having the potential to destroy the tumor can be conjugated to the anti-CD20 antibody such that the agent is specifically “delivered” to the neoplastic B cells. Irrespective of the approach, a primary goal is to destroy the tumor; the specific approach can be determined by the particular anti-CD20 antibody that is utilized, and thus, the available approaches to targeting the CD20 antigen can vary considerably.

The rituximab (RITUXAN®) antibody is a genetically engineered chimeric murine/human monoclonal antibody directed against the CD20 antigen. Rituximab is the antibody called “C2B8” in U.S. Pat. No. 5,736,137 issued Apr. 7, 1998 (Anderson et al.). Rituximab is indicated for the treatment of patients with relapsed or refractory low-grade or follicular, CD20-positive, B-cell non-Hodgkin's lymphoma. In vitro mechanism-of-action studies have demonstrated that rituximab binds human complement and lyses lymphoid B-cell lines through complement-dependent cytotoxicity (CDC). Reff et al., Blood 83(2):435-445 (1994). Additionally, it has significant activity in assays for antibody-dependent cellular cytotoxicity (ADCC). More recently, rituximab has been shown to have anti-proliferative effects in tritiated thymidine-incorporation assays and to induce apoptosis directly, while other anti-CD19 and anti-CD20 antibodies do not. Maloney et al. Blood 88(10):637a (1996). Synergy between rituximab and chemotherapies and toxins has also been observed experimentally. In particular, rituximab sensitizes drug-resistant human B-cell lymphoma cell lines to the cytotoxic effects of doxorubicin, CDDP, VP-16, diphtheria toxin, and ricin (Demidem et al., Cancer Chemotherapy & Radiopharmaceuticals 12(3): 177-186 (1997)). In vivo preclinical studies have shown that rituximab depletes B cells from the peripheral blood, lymph nodes, and bone marrow of cynomolgus monkeys, presumably through complement- and cell-mediated processes. Reff et al., Blood 83:435-445 (1994).

Rituximab was approved in the United States in November 1997 for the treatment of patients with relapsed or refractory low-grade or follicular CD20⁺ B-cell NHL at a dose of 375 mg/m² weekly for four doses. In April 2001, the Food and Drug Administration (FDA) approved additional claims for the treatment of low-grade NHL: re-treatment (weekly for four doses) and an additional dosing regimen (weekly for eight doses). There have been more than 300,000 patient exposures to rituximab either as monotherapy or in combination with immunosuppressant or chemotherapeutic drugs. Patients have also been treated with rituximab as maintenance therapy for up to 2 years. Hainsworth et al., J. Clin. Oncol. 21:1746-1751 (2003); Hainsworth et al., J. Clin. Oncol. 20:4261-4267 (2002). Also, rituximab has been used in the treatment of malignant and nonmalignant plasma cell disorders. Treon and Anderson, Semin. Oncol. 27: 79-85 (2000).

Rituximab has also been studied in a variety of non-malignant autoimmune disorders, in which B cells and autoantibodies appear to play a role in disease pathophysiology. Edwards et al., Biochem Soc. Trans. 30:824-828 (2002). Rituximab has been reported to potentially relieve signs and symptoms of, for example, rheumatoid arthritis (RA) (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Shaw et al. Ann. Rheum. Dis. 62 Suppl 2:ii55-ii59 (2003); Weyand and Goronzy, Ann. N.Y. Acad. Sci. 987: 140-149 (2003); Emery et al., Arthritis Rheum. 48(9): S439 (2003)), lupus (Eisenberg, Arthritis. Res. Ther. 5:157-159 (2003); Anolik et al., Arthritis Rheum. 48: 455-459 (2003); Leandro et al. Arthritis Rheum. 46: 2673-2677 (2002); Gorman et al., Lupus, 13: 312-316 (2004); Tomietto et al., Thromb. Haemost. 92: 1150-1153 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaja et al., Haematologica, 87: 189-195 (2002); Zaja et al., Haematologica 88: 538-546 (2003); Cooper et al., Br. J. Haematol. 125: 232-239 (2004); Ratanatharathorn et al., Ann. Int. Med., 133: 275-279 (2000)), pure red cell aplasia (Auner et al., Br. J. Haematol., 116: 725-728 (2002)); autoimmune anemia (Zaja et al., Haematologica 87:189-195 (2002) (erratum appears in Haematologica 87:336 (2002); Raj et al., J. Pediatr. Hematol. Oncol. 26: 312-314 (2004); Zecca et al., Blood 101: 3857-3861 (2003); Quartier et al., Lancet 358: 1511-1513 (2001)), autoimmune cytopenias (Robak, Eur. J. Haematol. 72: 79-88 (2004)); cold agglutinin disease (Layios et al., Leukemia, 15: 187-8 (2001); Berentsen et al., Blood, 103: 2925-2928 (2004); Berentsen et al., Br. J. Haematol., 115: 79-83 (2001); Bauduer, Br. J. Haematol., 112: 1083-1090 (2001); Damiani et al., Br. J. Haematol., 114: 229-234 (2001); Lee and Kueck, Blood 92: 3490-3491 (1998)), type B syndrome of severe insulin resistance (Coll et al., N. Engl. J. Med., 350: 310-311 (2004), mixed cryoglobulinemia (DeVita et al., Arthritis Rheum. 46 Suppl. 9:S206/S469 (2002); Zaja et al. Haematologica 84: 1157-1158 (1999)), myasthenia gravis (Zaja et al., Neurology, 55: 1062-63 (2000); Wylam et al., J. Pediatr., 143: 674-677 (2003)), Wegener's granulomatosis (Specks et al., Arthritis & Rheumatism 44: 2836-2840 (2001)), refractory pemphigus vulgaris (Dupuy et al., Arch Dermatol., 140:91-96 (2004)), dermatomyositis (Levine, Arthritis Rheum., 46 (Suppl. 9):S1299 (2002)), Sjogren's syndrome (Somer et al., Arthritis & Rheumatism, 49: 394-398 (2003)), active type-II mixed cryoglobulinemia (Zaja et al., Blood, 101: 3827-3834 (2003)), pemphigus vulgaris (Dupay et al., Arch. Dermatol., 140: 91-95 (2004)), autoimmune neuropathy (Pestronk et al., J. Neurol. Neurosurg. Psychiatry 74:485-489 (2003); Nobile-Orazio, Curr. Opin. Neurol. 17: 599-605 (2004); Rojas-Garcia et al., Neurology 61: 1814-1816 (2003); Renaud et al. Muscle Nerve 27: 611-615 (2003)), paraneoplastic opsoclonus-myoclonus syndrome (Pranzatelli et al. Neurology 60(Suppl. 1) PO5.128:A395 (2003)), acquired factor VIII inhibitors (Wiestner et al. Blood 100: 3426-3428 (2002); and relapsing-remitting multiple sclerosis (RRMS). Cross et al. (abstract) “Preliminary Results from a Phase II Trial of Rituximab in MS” Eighth Annual Meeting of the Americas Committees for Research and Treatment in Multiple Sclerosis, 20-21 (2003).

A Phase II study (WA16291) has been conducted in patients with rheumatoid arthritis (RA), providing 48-week follow-up data on safety and efficacy of Rituximab. Emery et al. Arthritis Rheum 48(9):S439 (2003); Szczepanski et al. Arthritis Rheum 48(9):S121 (2003). A total of 161 patients were evenly randomized to four treatment arms: methotrexate, rituximab alone, rituximab plus methotrexate, and rituximab plus cyclophosphamide (CTX). The treatment regimen of rituximab was one gram administered intravenously on days 1 and 15. Infusions of rituximab in most patients with RA were well tolerated by most patients, with 36% of patients experiencing at least one adverse event during their first infusion (compared with 30% of patients receiving placebo). Overall, the majority of adverse events was considered to be mild to moderate in severity and was well balanced across all treatment groups. There were a total of 19 serious adverse events across the four arms over the 48 weeks, which were slightly more frequent in the rituximab/CTX group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study. Doran et al., Arthritis Rheum. 46:2287-2293 (2002).

The reported safety profile of rituximab in a small number of patients with neurologic disorders, including autoimmune neuropathy (Pestronk et al., supra), opsoclonus-myoclonus syndrome (Pranzatelli et al., supra), and RRMS (Cross et al., supra), was similar to that reported in oncology or RA. In an ongoing investigator-sponsored trial (IST) of rituximab in combination with interferon-beta (IFN-β) or glatiramer acetate in patients with RRMS (Cross et al., supra), 1 of 10 treated patients was admitted to the hospital for overnight observation after experiencing moderate fever and rigors following the first infusion of rituximab, while the other 9 patients completed the four-infusion regimen without any reported adverse events.

Patents and patent publications concerning CD20 antibodies and CD20-binding molecules include U.S. Pat. Nos. 5,776,456, 5,736,137, 5,843,439, 6,399,061, and 6,682,734, as well as US 2002/0197255, US 2003/0021781, US 2003/0082172, US 2003/0095963, US 2003/0147885 (Anderson et al.); U.S. Pat. No. 6,455,043 and WO 2000/09160 (Grillo-Lopez, A.); WO 2000/27428 (Grillo-Lopez and White); WO 2000/27433 (Grillo-Lopez and Leonard); WO 2000/44788 (Braslawsky et al.); WO 2001/10462 (Rastetter, W.); WO 2001/10461 (Rastetter and White); WO 2001/10460 (White and Grillo-Lopez); US 2001/0018041, US 2003/0180292, WO 2001/34194 (Hanna and Hariharan); US 2002/0006404 and WO 2002/04021 (Hanna and Hariharan); US 2002/0012665, WO 2001/74388 and 6,896,885B5 (Hanna, N.); US 2002/0058029 (Hanna, N.); US 2003/0103971 (Hariharan and Hanna); US 2005/0123540 (Hanna et al.); US 2002/0009444 and WO 2001/80884 (Grillo-Lopez, A.); WO 2001/97858; US 2005/0112060, and U.S. Pat. No. 6,846,476 (White, C.); US 2002/0128488 and WO 2002/34790 (Reff, M.); WO 2002/060955 (Braslawsky et al.); WO 2002/096948 (Braslawsky et al.); WO 2002/079255 (Reff and Davies); U.S. Pat. No. 6,171,586 and WO 1998/56418 (Lam et al.); WO 1998/58964 (Raju, S.); WO 1999/22764 (Raju, S.); WO 1999/51642, U.S. Pat. No. 6,194,551, U.S. Pat. No. 6,242,195, U.S. Pat. No. 6,528,624 and U.S. Pat. No. 6,538,124 (Idusogie et al.); WO 2000/42072 (Presta, L.); WO 2000/67796 (Curd et al.); WO 2001/03734 (Grillo-Lopez et al.); US 2002/0004587 and WO 2001/77342 (Miller and Presta); US 2002/0197256 (Grewal, I.); US 2003/0157108 (Presta, L.); U.S. Pat. Nos. 6,565,827, 6,090,365, 6,287,537, 6,015,542, 5,843,398, and 5,595,721, (Kaminski et al.); U.S. Pat. Nos. 5,500,362, 5,677,180, 5,721,108, 6,120,767, 6,652,852, 6,893,625 (Robinson et al.); U.S. Pat. No. 6,410,391 (Raubitschek et al.); U.S. Pat. No. 6,224,866 and WO00/20864 (Barbera-Guillem, E.); WO 2001/13945 (Barbera-Guillem, E.); WO 2000/67795 (Goldenberg); US 2003/0133930 and WO 2000/74718 (Goldenberg and Hansen); US 2003/0219433 and WO 2003/68821 (Hansen et al.); WO 2004/058298 (Goldenberg and Hansen); WO 2000/76542 (Golay et al.); WO 2001/72333 (Wolin and Rosenblatt); U.S. Pat. No. 6,368,596 (Ghetie et al.); U.S. Pat. No. 6,306,393 and US 2002/0041847 (Goldenberg, D.); US 2003/0026801 (Weiner and Hartmann); WO 2002/102312 (Engleman, E.); US 2003/0068664 (Albitar et al.); WO 2003/002607 (Leung, S.); WO 2003/049694, US 2002/0009427, and US 2003/0185796 (Wolin et al.); WO 2003/061694 (Sing and Siegall); US 2003/0219818 (Bohen et al.); US 2003/0219433 and WO 2003/068821 (Hansen et al.); US 2003/0219818 (Bohen et al.); US 2002/0136719 (Shenoy et al.); WO 2004/032828 and US 2005/0180972 (Wahl et al.); and WO 2002/56910 (Hayden-Ledbetter). See also U.S. Pat. No. 5,849,898 and EP 330,191 (Seed et al.); EP332,865A2 (Meyer and Weiss); U.S. Pat. No. 4,861,579 (Meyer et al.); US 2001/0056066 (Bugelski et al.); WO 1995/03770 (Bhat et al.); US 2003/0219433 A1 (Hansen et al.); WO 2004/035607 (Teeling et al.); WO 2004/056312 (Lowman et al.); US 2004/0093621 (Shitara et al.); WO 2004/103404 (Watkins et al.); WO 2005/000901 (Tedder et al.); US 2005/0025764 (Watkins et al.); WO 2005/016969 (Carr et al.); US 2005/0069545 (Carr et al.); WO 2005/014618 (Chang et al.); US 2005/0079174 (Barbera-Guillem and Nelson); US 2005/0106108 (Leung and Hansen); WO2005/044859 and US 2005/0123546 (Umana et al.); WO 2005/070963 (Allan et al.); US 2005/0186216 (Ledbetter and Hayden-Ledbetter); and U.S. Pat. No. 6,897,044 (Braslawski et al.).

Publications concerning treatment with rituximab include: Perotta and Abuel, “Response of chronic relapsing ITP of 10 years duration to rituximab” Abstract # 3360 Blood 10(1)(part 1-2): p. 88B (1998); Perotta et al., “Rituxan in the treatment of chronic idiopathic thrombocytopaenic purpura (ITP)”, Blood, 94: 49 (abstract) (1999); Matthews, R., “Medical Heretics” New Scientist (7 April, 2001); Leandro et al., “Clinical outcome in 22 patients with rheumatoid arthritis treated with B lymphocyte depletion” Ann Rheum Dis, supra; Leandro et al., “Lymphocyte depletion in rheumatoid arthritis: early evidence for safety, efficacy and dose response” Arthritis and Rheumatism 44(9): S370 (2001); Leandro et al., “An open study of B lymphocyte depletion in systemic lupus erythematosus”, Arthritis and Rheumatism, 46:2673-2677 (2002), wherein during a 2-week period, each patient received two 500-mg infusions of rituximab, two 750-mg infusions of cyclophosphamide, and high-dose oral corticosteroids, and wherein two of the patients treated relapsed at 7 and 8 months, respectively, and have been retreated, although with different protocols; “Successful long-term treatment of systemic lupus erythematosus with rituximab maintenance therapy” Weide et al., Lupus, 12: 779-782 (2003), wherein a patient was treated with rituximab (375 mg/m²×4, repeated at weekly intervals) and further rituximab applications were delivered every 5-6 months and then maintenance therapy was received with rituximab 375 mg/m² every three months, and a second patient with refractory SLE was treated successfully with rituximab and is receiving maintenance therapy every three months, with both patients responding well to rituximab therapy; Edwards and Cambridge, “Sustained improvement in rheumatoid arthritis following a protocol designed to deplete B lymphocytes” Rheumatology 40:205-211 (2001); Cambridge et al., “B lymphocyte depletion in patients with rheumatoid arthritis: serial studies of immunological parameters” Arthritis Rheum., 46 (Suppl. 9): S1350 (2002); Cambridge et al., “Serologic changes following B lymphocyte depletion therapy for rheumatoid arthritis” Arthritis Rheum., 48: 2146-2154 (2003); Edwards et al., “B-lymphocyte depletion therapy in rheumatoid arthritis and other autoimmune disorders” Biochem Soc. Trans., supra; Edwards et al., “Efficacy and safety of rituximab, a B-cell targeted chimeric monoclonal antibody: A randomized, placebo controlled trial in patients with rheumatoid arthritis. Arthritis and Rheumatism 46(9): S197 (2002); Edwards et al., “Efficacy of B-cell-targeted therapy with rituximab in patients with rheumatoid arthritis” N Engl. J. Med. 350:2572-2582 (2004); Pavelka et al., Ann. Rheum. Dis. 63: (S1):289-290 (2004); Emery et al., Arthritis Rheum. 50 (S9):S659 (2004); Levine and Pestronk, “IgM antibody-related polyneuropathies: B-cell depletion chemotherapy using rituximab” Neurology 52: 1701-1704 (1999); Uchida et al., “The innate mononuclear phagocyte network depletes B lymphocytes through Fc receptor-dependent mechanisms during anti-CD20 antibody immunotherapy” J. Exp. Med. 199: 1659-1669 (2004); Gong et al., “Importance of cellular microenvironment and circulatory dynamics in B cell immunotherapy” J. Immunol. 174: 817-826 (2005); Hamaguchi et al., “The peritoneal cavity provides a protective niche for B1 and conventional B lymphocytes during anti-CD20 immunotherapy in mice” J. Immunol. 174: 4389-4399 (2005); Cragg et al. “The biology of CD20 and its potential as a target for mAb therapy” Curr. Dir. Autoimmun. 8:140-174 (2005); Eisenberg, “Mechanisms of autoimmunity” Immunol. Res. 27: 203-218 (2003); DeVita et al., “Efficacy of selective B cell blockade in the treatment of rheumatoid arthritis” Arthritis & Rheum 46:2029-2033 (2002); Hidashida et al. “Treatment of DMARD-refractory rheumatoid arthritis with rituximab.” Presented at the Annual Scientific Meeting of the American College of Rheumatology; October 24-29; New Orleans, La. 2002; Tuscano, J. “Successful treatment of infliximab-refractory rheumatoid arthritis with rituximab” Presented at the Annual Scientific Meeting of the American College of Rheumatology; October 24-29; New Orleans, La. 2002 and published Tuscano, Arthritis Rheum. 46: 3420 (2002); “Pathogenic roles of B cells in human autoimmunity; insights from the clinic” Martin and Chan, Immunity 20:517-527 (2004); Silverman and Weisman, “Rituximab therapy and autoimmune disorders, prospects for anti-B cell therapy”, Arthritis and Rheumatism, 48: 1484-1492 (2003); Kazkaz and Isenberg, “Anti B cell therapy (rituximab) in the treatment of autoimmune diseases”, Current opinion in pharmacology, 4: 398-402 (2004); Virgolini and Vanda, “Rituximab in autoimmune diseases”, Biomedicine & pharmacotherapy, 58: 299-309(2004); Klemmer et al., “Treatment of antibody mediated autoimmune disorders with a AntiCD20 monoclonal antibody Rituximab”, Arthritis And Rheumatism, 48: (9) 9,S (SEP), page: S624-S624 (2003); Kneitz et al., “Effective B cell depletion with rituximab in the treatment of autoimmune diseases”, Immunobiology, 206: 519-527 (2002); Arzoo et al., “Treatment of refractory antibody mediated autoimmune disorders with an anti-CD20 monoclonal antibody (rituximab)” Annals of the Rheumatic Diseases, 61 (10), p 922-924 (2002) Comment in Ann Rheum Dis. 61: 863-866 (2002); “Future strategies in immunotherapy” by Lake and Dionne, in Burger's Medicinal Chemistry and Drug Discovery (2003 by John Wiley & Sons, Inc.) Article Online Posting Date: Jan. 15, 2003 (Chapter 2 “Antibody-Directed Immunotherapy”); Liang and Tedder, Wiley Encyclopedia of Molecular Medicine, Section: CD20 as an Immunotherapy Target, article online posting date: 15 Jan., 2002 entitled “CD20”; Appendix 4A entitled “Monoclonal Antibodies to Human Cell Surface Antigens” by Stockinger et al., eds: Coligan et al., in Current Protocols in Immunology (2003 John Wiley & Sons, Inc) Online Posting Date: May, 2003; Print Publication Date: February, 2003; Penichet and Morrison, “CD Antibodies/molecules: Definition; Antibody Engineering” in Wiley Encyclopedia of Molecular Medicine Section: Chimeric, Humanized and Human Antibodies; posted online 15 January, 2002.

Further, see Looney “B cells as a therapeutic target in autoimmune diseases other than rheumatoid arthritis” Rheumatology, 44 Suppl 2: ii13-ii17 (2005); Chambers and Isenberg, “Anti-B cell therapy (rituximab) in the treatment of autoimmune diseases” Lupus 14(3): 210-214 (2005); i Looney et al., “B-cell depletion as a novel treatment for systemic lupus erythematosus: a phase I/II dose-escalating trial of rituximab” Arthritis Rheum. 50: 2580-2589 (2004); Looney, “Treating human autoimmune disease by depleting B cells” Ann Rheum. Dis. 61: 863-866 (2002); Edelbauer et al., “Rituximab in childhood systemic lupus erythematosus refractory to conventional immunosuppression Case report” Pediatr. Nephrol. 20(6): 811-813 (2005); D'Cruz and Hughes, “The treatment of lupus nephritis” BMJ 330(7488): 377-378 (2005); Looney, “B cell-targeted therapy in diseases other than rheumatoid arthritis” J. Rheumatol. Suppl. 73: 25-28; discussion 29-30 (2005); Sfikakis et al., “Remission of proliferative lupus nephritis following B cell depletion therapy is preceded by down-regulation of the T cell costimulatory molecule CD40 ligand: an open-label trial” Arthritis Rheum. 52(2): 501-513 (2005); Rastetter et al., “Rituximab: expanding role in therapy for lymphomas and autoimmune diseases” Annu. Rev. Med. 55: 477-503 (2004); Silverman, “Anti-CD20 therapy in systemic lupus erythematosus: a step closer to the clinic” Arthritis Rheum. 52(2): 371-7 (2005), Erratum in: Arthritis Rheum. 52(4): 1342 (2005); Ahn et al., “Long-term remission from life-threatening hypercoagulable state associated with lupus anticoagulant (LA) following rituximab therapy” Am. J. Hematol. 78(2): 127-129 (2005); Tahir et al., “Humanized anti-CD20 monoclonal antibody in the treatment of severe resistant systemic lupus erythematosus in a patient with antibodies against rituximab” Rheumatology, 44(4): 561-562 (2005), Epub 2005 Jan 11; Looney et al., “Treatment of SLE with anti-CD20 monoclonal antibody” Curr. Dir. Autoimmun. 8: 193-205 (2005); Cragg et al., “The biology of CD20 and its potential as a target for mAb therapy” Curr. Dir. Autoimmun. 8: 140-174 (2005); Gottenberg et al., “Tolerance and short term efficacy of rituximab in 43 patients with systemic autoimmune diseases” Ann. Rheum. Dis. 64(6): 913-920 (2005) Epub 2004 Nov. 18; Tokunaga et al., “Down-regulation of CD40 and CD80 on B cells in patients with life-threatening systemic lupus erythematosus after successful treatment with rituximab” Rheumatology 44(2): 176-182 (2005), Epub 2004 Oct. 19. See also Leandro et al., “B cell repopulation occurs mainly from naïve B cells in patient with rheumatoid arthritis and systemic lupus erythematosus” Arthritis Rheum., 48 (Suppl 9): S1160 (2003).

Specks et al. “Response of Wegener's granulomatosis to anti-CD20 chimeric monoclonal antibody therapy” Arthritis & Rheumatism 44(12):2836-2840 (2001) discloses successful use of four infusions of 375 mg/m² of rituximab and high-dose glucocorticoids to treat Wegener's granulomatosis. The therapy was repeated after 11 months when the cANCA recurred, but therapy was without glucocorticoids. At 8 months after the second course of rituximab, the patients' disease remained in complete remission. Further, in another study, rituximab was found to be a well-tolerated, effective remission induction agent for severe ANCA-associated vasculitis, when used in a dose of 375 mg/m²×4 along with oral prednisone 1 mg/kg/day, which was reduced by week 4 to 40 mg/day, and to complete discontinuation over the following 16 weeks. Four patients were re-treated with rituximab alone for recurring/rising ANCA titers. Other than glucocorticoids, no additional immunosuppressive agents seem to be necessary for remission induction and maintenance of sustained remission (6 months or longer). See online abstract submission and invitation Keogh et al., “Rituximab for Remission Induction in Severe ANCA-Associated Vasculitis: Report of a Prospective Open-Label Pilot Trial in 10 Patients”, American College of Rheumatology, Session Number: 28-100, Session Title: Vasculitis, Session Type: ACR Concurrent Session, Primary Category: 28 Vasculitis, Session Oct. 18, 2004 (<www.abstractsonline.com/viewer/SearchResults.asp>). See also Keogh et al., Kidney Blood Press. Res. 26:293 (2003), wherein it is reported that eleven patients with refractory ANCA-associated vasculitis were treated with four weekly doses of 375 mg/m² of rituximab and high-dose glucocortoicoids, resulting in remission.

Patients with refractory ANCA-associated vasculitis were administered rituximab along with immunosuppressive medicaments such as intravenous cyclophosphamide, mycophenolate mofetil, azathioprine, or leflunomide, with apparent efficacy. Eriksson, “Short-term outcome and safety in 5 patients with ANCA-positive vasculitis treated with rituximab”, Kidney and Blood Pressure Research, 26: 294 (2003) (five patients with ANCA-associated vasculitis treated with rituximab 375 mg/m² once a week for 4 weeks responded to the treatment); Jayne et al., “B-cell depletion with rituximab for refractory vasculitis” Kidney and Blood Pressure Research, 26: 294-295 (2003) (six patients with refractory vasculitis receiving four weekly infusions of rituximab at 375 mg/m² with cyclophosphamide along with background immunosuppression and prednisolone experienced major falls in vasculitic activity). A further report of using rituximab along with intravenous cyclophosphamide at 375 mg/m² per dose in 4 doses for administering to patients with refractory systemic vasculitis is provided in Jayne, poster 88 (11^{th} International Vasculitis and ANCA workshop), 2003 American Society of Nephrology. See also Stone and Specks, “Rituximab Therapy for the Induction of Remission and Tolerance in ANCA-associated Vasculitis”, in the Clinical Trial Research Summary of the 2002-2003 Immune Tolerance Network, http://www.immunetolerance.org/research/autoimmune/trials/stone.html, in which a trial of rituximab in ANCA-associated vasculitis is proposed for a total length of 18 months. See also Eriksson, J. Internal Med., 257: 540-548 (2005) regarding nine patients with ANCA-positive vasculitis who were successfully treated with two or four weekly doses of 500 mg of rituximab, as well as Keogh et al., Arthritis and Rheumatism, 52: 262-268 (2005), who reported that in 11 patients with refractory ANCA-associated vasculitis, treatment or re-treatment with four weekly doses of 375 mg/m² of rituximab induced remission by B lymphocyte depletion, the study being conducted between January 2000 and September 2002.

As to the activity of a humanized anti-CD20 antibody, see, for example, Vugmeyster et al., “Depletion of B cells by a humanized anti-CD20 antibody PRO70769 in Macaca fascicularis” J. Immunother. 28: 212-219 (2005). For discussion of a human monoclonal antibody, see Baker et al., “Generation and characterization of LymphoStat-B, a human monoclonal antibody that antagonizes the bioactivities of B lymphocyte stimulator” Arthritis Rheum. 48: 3253-3265 (2003)

There remains a need for approaches to treatment that reduce the frequency of infusions of active drug within a month's time. Further, there is a need to reduce the risk of toxic effects of currently used drugs such as steroids and chemotherapeutic agents, and to reduce the risk of disease flares, relapses, and recurrences in patients with ANCA-associated vasculitis, and to sustain remission and maintain sustained remission for a prolonged period of time.

### SUMMARY OF THE INVENTION

The present invention involves administration of a CD20 antibody that provides a safe and active treatment regimen in subjects with ANCA-associated vasculitis, including selection of an efficacious dosing regimen and scheduled or unscheduled re-treatment. This antagonist is effective both in initial therapy and in the management of refractory disease.

Accordingly, the invention is as claimed. In a first aspect, the present invention concerns treating ANCA-associated vasculitis in a patient comprising administering a CD20 antibody to the patient in a dose of about 400 mg to 1.3 grams at a frequency of one to three doses within a period of about one month.

In a further aspect, the invention provides an article of manufacture comprising: a container comprising a CD20 antibody and a package insert with instructions for treating ANCA-associated vasculitis in a patient, wherein the instructions indicate that a dose of the CD20 antibody of about 400 mg to 1.3 grams, at a frequency of one to three doses, is administered to the patient within a period of about one month.

In preferred embodiments of the above inventive aspects, the vasculitis is Wegener's granulomatosis or microscopic polyangiitis, and/or a second medicament is administered in an effective amount to the patient, wherein the CD20 antibody is a first medicament. Such medicament may be one or more medicaments. More preferably, such second medicament is a chemotherapeutic agent, an immunosuppressive agent, a disease-modifying anti-rheumatic drug (DMARD), a cytotoxic agent, an integrin antagonist, a non-steroidal anti-inflammatory drug (NSAID), a cytokine antagonist, a hormone, or a combination thereof.

In still further aspects, the present invention relates to a method of treating ANCA-associated vasculitis in a subject comprising administering an effective amount of a CD20 antibody to the subject to provide an initial antibody exposure followed by a second antibody exposure, wherein the second exposure is not provided until from about 16 to 54 weeks from the initial exposure.

In one preferred embodiment of this lattermost method involving multiple antibody exposures, the present invention relates to a method of treating ANCA-associated vasculitis in a subject comprising administering to the subject an effective amount of a CD20 antibody to provide an initial antibody exposure of about 0.5 to 4 grams followed by a second antibody exposure of about 0.5 to 4 grams, wherein the second exposure is not provided until from about 16 to 54 weeks from the initial exposure and each of the antibody exposures is provided to the subject as about 1 to 4 doses of antibody, more preferably as a single dose or as two or three separate doses of antibody.

A specific preferred embodiment herein is a method of treating ANCA-associated vasculitis in a subject comprising administering an effective amount of a CD20 antibody to the subject to provide an initial antibody exposure followed by a second antibody exposure, wherein the second exposure is not provided until from about 16 to 54 weeks from the initial exposure and each of the antibody exposures is provided to the subject as a single dose or as two or three separate doses of antibody. Preferably in such a method, the antibody exposures are of about 0.5 to 4 grams each.

In another preferred embodiment of these lattermost methods, a second medicament is administered with the initial exposure and/or later exposures, wherein the antibody is a first medicament. In a preferred embodiment, the second medicament is one or more of those set forth above as preferred. In a more preferred embodiment, the second medicament is a steroid and/or an immunosuppressive agent. In a still preferred embodiment, a steroid is administered with the first exposure, but not with the second exposure, or is administered in lower amounts than are used with the initial exposure.

In still another preferred embodiment of these lattermost aspects, the subject has never been previously treated with a CD20 antibody, and/or no other medicament than the CD20 antibody is administered to the subject to treat the vasculitis. In another preferred embodiment, the initial and second antibody exposures are with the same antibody, and more preferably all antibody exposures are with the same antibody. In another preferred embodiment, the subject is in remission after the initial or later antibody exposures, preferably when provided the second antibody exposure. More preferably, the subject is in remission when provided all antibody exposures. Most preferably, such subject is in remission at least about six months after the last antibody exposure provided.

In yet another preferred embodiment of these lattermost aspects, the subject has an elevated level of anti-nuclear antibodies (ANA), anti-rheumatoid factor (RF) antibodies, creatinine, blood urea nitrogen, anti-endothelial antibodies, anti-neutrophil cytoplasmic antibodies (ANCA), or a combination of two or more thereof.

Additionally, in further aspects, the invention provides an article of manufacture comprising:

(a) a container comprising a CD20 antibody; and

(b) a package insert with instructions for treating ANCA-associated vasculitis in a subject, wherein the instructions indicate that an amount of the antibody is administered to the subject that is effective to provide an initial antibody exposure followed by a second antibody exposure, wherein the second exposure is not provided until from about 16 to 54 weeks from the initial exposure.

Preferably, such package insert is provided with instructions for treating ANCA-associated vasculitis in a subject, wherein the instructions indicate that an amount of the antibody is administered to the subject that is effective to provide an initial antibody exposure of about 0.5 to 4 grams followed by a second antibody exposure of about 0.5 to 4 grams, wherein the second exposure is not provided until from about 16 to 54 weeks from the initial exposure and each of the antibody exposures is provided to the subject as about one to four doses, preferably as a single dose or as two or three separate doses of antibody.

In a specific aspect, an article of manufacture is provided comprising:

(a) a container comprising a CD20 antibody; and

(b) a package insert with instructions for treating ANCA-associated vasculitis in a subject, wherein the instructions indicate that an amount of the antibody is administered to the subject that is effective to provide an initial antibody exposure followed by a second antibody exposure, wherein the second exposure is not provided until from about 16 to 54 weeks from the initial exposure, and each of the antibody exposures is provided to the subject as a single dose or as two or three separate doses of antibody.

The treatments herein preferably reduce, minimize, or eliminate the need for co-, pre-, or post-administration of excessive amounts of second medicaments such as immunosuppressive agents and/or chemotherapeutic agents that are ordinarily standard treatment for such subjects, to avoid as much as possible the side effects of such standard treatment, as well as reduce costs and increase convenience to the subject, such as convenience of time and frequency of administration.

### BRIEF DESCRIPTION OF THE DRAWINGS

FIG. 1A is a sequence alignment comparing the amino acid sequences of the light chain variable domain (V_{L}) of each of murine 2H7 (SEQ ID NO:1), humanized 2H7.v16 variant (SEQ ID NO:2), and the human kappa light chain subgroup I (SEQ ID NO:3). The CDRs of V_{L} of 2H7 and hu2H7.v16 are as follows: CDR1 (SEQ ID NO:4), CDR2 (SEQ ID NO:5), and CDR3 (SEQ ID NO:6).

FIG. 1B is a sequence alignment comparing the amino acid sequences of the heavy chain variable domain (V_{H}) of each of murine 2H7 (SEQ ID NO:7), humanized 2H7.v16 variant (SEQ ID NO:8), and the human consensus sequence of the heavy chain subgroup III (SEQ ID NO:9). The CDRs of V_{H} of 2H7 and hu2H7.v16 are as follows: CDR1 (SEQ ID NO:10), CDR2 (SEQ ID NO:11), and CDR3 (SEQ ID NO:12).

In FIG. 1A and FIG. 1B, the CDR1, CDR2 and CDR3 in each chain are enclosed within brackets, flanked by the framework regions, FR1-FR4, as indicated. 2H7 refers to the murine 2H7 antibody. The asterisks in between two rows of sequences indicate the positions that are different between the two sequences. Residue numbering is according to Kabat et al. Sequences of Immunological Interest, 5th Ed. Public Health Service, National Institutes of Health, Bethesda, Md. (1991), with insertions shown as a, b, c, d, and e.

FIG. 2 shows the amino acid sequence of the mature 2H7.v16 L chain (SEQ ID NO:13)

FIG. 3 shows the amino acid sequence of the mature 2H7.v16H chain (SEQ ID NO:14).

FIG. 4 shows the amino acid sequence of the mature 2H7.v31H chain (SEQ ID NO:15). The L chain of 2H7.v31 is the same as for 2H7.v16.

FIG. 5 is a sequence alignment comparing the light-chain amino acid sequences of the humanized 2H7.v16 variant (SEQ ID NO:2) and humanized 2H7.v138 variant (SEQ ID NO:28).

FIG. 6 is a sequence alignment comparing the heavy-chain amino acid sequences of the humanized 2H7.v16 variant (SEQ ID NO:8) and humanized 2H7.v138 variant (SEQ ID NO:29).

FIG. 7 shows an alignment of the mature 2H7.v16 and 2H7.v511 light chains (SEQ ID NOS: 13 and 30, respectively), with Kabat variable-domain residue numbering and Eu constant-domain residue numbering.

FIG. 8 shows an alignment of the mature 2H7.v16 and 2H7.v511 heavy chains (SEQ ID NOS:14 and 31, respectively), with Kabat variable-domain residue numbering and Eu constant-domain residue numbering.

FIG. 9A shows the sequence of the humanized 2H7.v114 variable light-chain domain (SEQ ID NO:32); FIG. 9B shows the sequence of the humanized 2H7.v114 variable heavy-chain domain (SEQ ID NO:33); and FIG. 9C shows the sequence of the humanized 2H7.v114 full-length heavy chain (SEQ ID NO:34), with Kabat variable-domain residue numbering and Eu constant-domain residue numbering.

### DETAILED DESCRIPTION OF THE PREFERRED EMBODIMENTS

I. Definitions

A “B cell” is a lymphocyte that matures within the bone marrow, and includes a naïve B cell, memory B cell, or effector B cell (plasma cells). The B cell herein may be a normal or non-malignant B cell.

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, B-cell CR2, CCR6, P2X5, HLA-DOB, CXCR5, FCER2, BR3, Btig, NAG14, SLGC16270, FcRH1, IRTA2, ATWD578, FcRH3, IRTA1, FcRH6, BCMA, and 239287. The B-cell surface marker of particular interest is preferentially expressed on B cells compared to other non-B-cell tissues of a mammal and may be expressed on both precursor B cells and mature B cells. The preferred B-cell surface markers herein are CD20 and CD22.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al., Proc. Natl. Acad. Sci. (USA) 82:1766 (1985), for example.

The “CD22” antigen, or “CD22,” also known as BL-CAM or Lyb8, is a type 1 integral membrane glycoprotein with molecular weight of about 130 (reduced) to 140 kD (unreduced). It is expressed in both the cytoplasm and cell membrane of B-lymphocytes. CD22 antigen appears early in B-cell lymphocyte differentiation at approximately the same stage as the CD19 antigen. Unlike other B-cell markers, CD22 membrane expression is limited to the late differentiation stages comprised between mature B cells (CD22+) and plasma cells (CD22−). The CD22 antigen is described, for example, in Wilson et al., J. Exp. Med. 173:137 (1991) and Wilson et al., J. Immunol. 150:5013 (1993).

An “antagonist” is a molecule that, upon binding to CD20 on B cells, destroys or depletes B cells in a mammal and/or interferes with one or more B cell functions, e.g. by reducing or preventing a humoral response elicited by the B cell. The antagonist preferably is able to deplete B cells (i.e. reduce circulating B cell levels) in a mammal treated therewith. Such depletion may be achieved via various mechanisms such antibody-dependent cell-mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native-sequence peptides, immunoadhesins, and small-molecule antagonists that bind to CD20, optionally conjugated with or fused to a cytotoxic agent. The preferred antagonist comprises an antibody.

An “antibody antagonist” herein is an antibody that, upon binding to a B-cell surface marker on B cells, destroys or depletes B cells in a mammal and/or interferes with one or more B-cell functions, e.g., by reducing or preventing a humoral response elicited by the B cell. The antibody antagonist preferably is able to deplete B cells (i.e., reduce circulating B-cell levels) in a mammal treated therewith. Such depletion may be achieved via various mechanisms such antibody-dependent cell-mediated cytotoxicity (ADCC) and/or complement-dependent cytotoxicity (CDC), inhibition of B-cell proliferation and/or induction of B-cell death (e.g., via apoptosis).

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

For the purposes herein, an “intact antibody” is one comprising heavy and light variable domains as well as an Fc region.

An “antibody that binds to a B-cell surface marker” is a molecule that, upon binding to a B-cell surface marker, destroys or depletes B cells in a mammal and/or interferes with one or more B-cell functions, e.g. by reducing or preventing a humoral response elicited by the B cell. The antibody preferably is able to deplete B cells (i.e. reduce circulating B-cell levels) in a mammal treated therewith. Such depletion may be achieved via various mechanisms such antibody-dependent cell-mediated cytotoxicity (ADCC) and/or complement-dependent cytotoxicity (CDC), inhibition of B-cell proliferation and/or induction of B-cell death (e.g. via apoptosis). In one preferred embodiment, the antibody induces a major clinical response. In another preferred embodiment, the B-cell surface marker is CD20, so that the antibody that binds to a B-cell surface marker is an antibody that binds to CD20, or a “CD20 antibody.” A particularly preferred embodiment is a CD20 antibody that induces a major clinical response. For purposes herein, a “major clinical response” is defined as achieving an American College of Rheumatology 70 response (ACR 70) for six consecutive months. ACR response scores are categorized as ACR 20, ACR 50 and ACR 70 with ACR 70 being the highest level of sign and symptom control in this evaluation system. ACR response scores measure improvement in rheumatoid arthritis disease activity, including joint swelling and tenderness, pain, level of disability and overall patient and physician assessment. An example of a different type of antibody that induces a major clinical response as recognized by the FDA and as defined herein is etanercept (ENBREL®),

Examples of CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™) commercially available from Corixa (see, also, U.S. Pat. No. 5,595,721); murine monoclonal antibody “1F5” (Press et al. Blood 69(2):584-591 (1987) and variants thereof including “framework patched” or humanized 1F5 (WO 2003/002607, Leung, S.; ATCC deposit HB-96450); murine 2H7 and chimeric 2H7 antibody (U.S. Pat. No. 5,677,180); a humanized 2H7 (WO 2004/056312 (Lowman et al.) and as set forth below); HUMAX-CD20™ fully human, high-affinity antibody targeted at the CD20 molecule in the cell membrane of B-cells (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO04/035607 (Teeling et al.); AME-133™ antibodies (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1B3, B-C1 or NU-B2 available from the International Leukocyte Typing Workshop (Valentine et al., In: Leukocyte Typing II (McMichael, Ed., p. 440, Oxford University Press (1987)). The preferred CD20 antibodies herein are chimeric, humanized, or human CD20 antibodies, more preferably rituximab, a humanized 2H7, chimeric or humanized A20 antibody (Immunomedics), and HUMAX-CD20™ human CD20 antibody (Genmab).

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof which retain the ability to bind CD20.

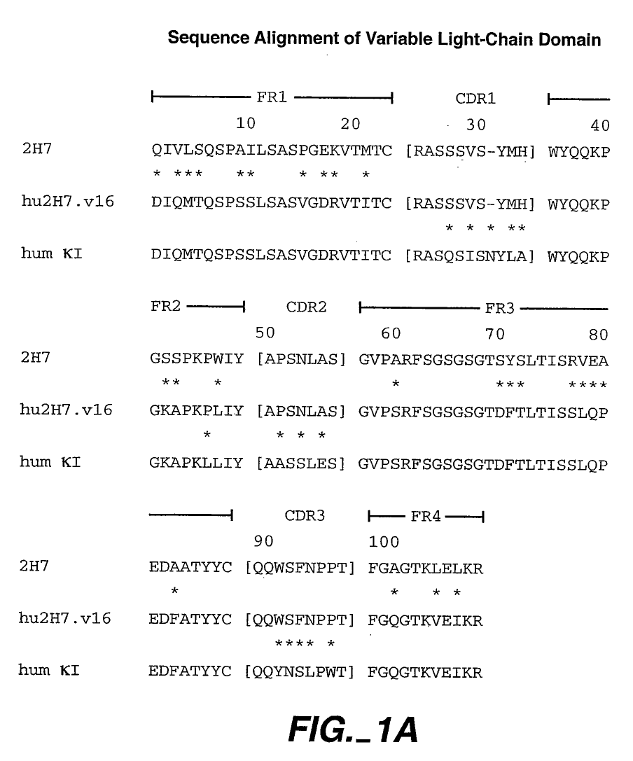

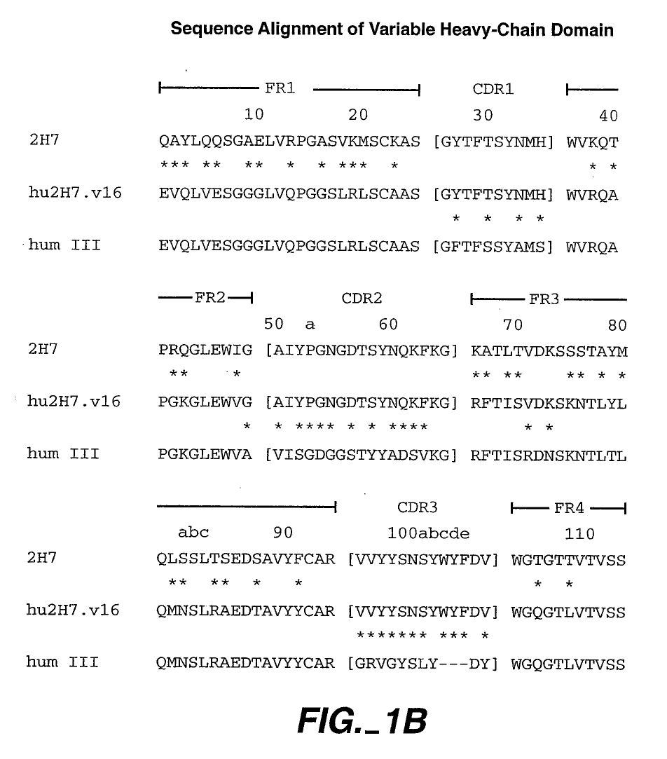

Purely for the purposes herein and unless indicated otherwise, a “humanized 2H7” refers to a humanized CD20 antibody, or an antigen-binding fragment thereof, wherein the antibody is effective to deplete primate B cells in vivo, the antibody comprising in the H chain variable region (V_{H}) thereof at least a CDR H3 sequence of SEQ ID NO:12 (FIG. 1B) from an anti-human CD20 antibody and substantially the human consensus framework (FR) residues of the human heavy-chain subgroup III (V_{H}III). In a preferred embodiment, this antibody further comprises the H chain CDR H1 sequence of SEQ ID NO:10 and CDR H2 sequence of SEQ ID NO:11, and more preferably further comprises the L chain CDR L1 sequence of SEQ ID NO:4, CDR L2 sequence of SEQ ID NO:5, CDR L3 sequence of SEQ ID NO:6 and substantially the human consensus framework (FR) residues of the human light chain subgroup I (VI), wherein the V_{H} region may be joined to a human IgG chain constant region, wherein the region may be, for example, IgG1 or IgG3. See also WO 2004/056312 (Lowman et al.).

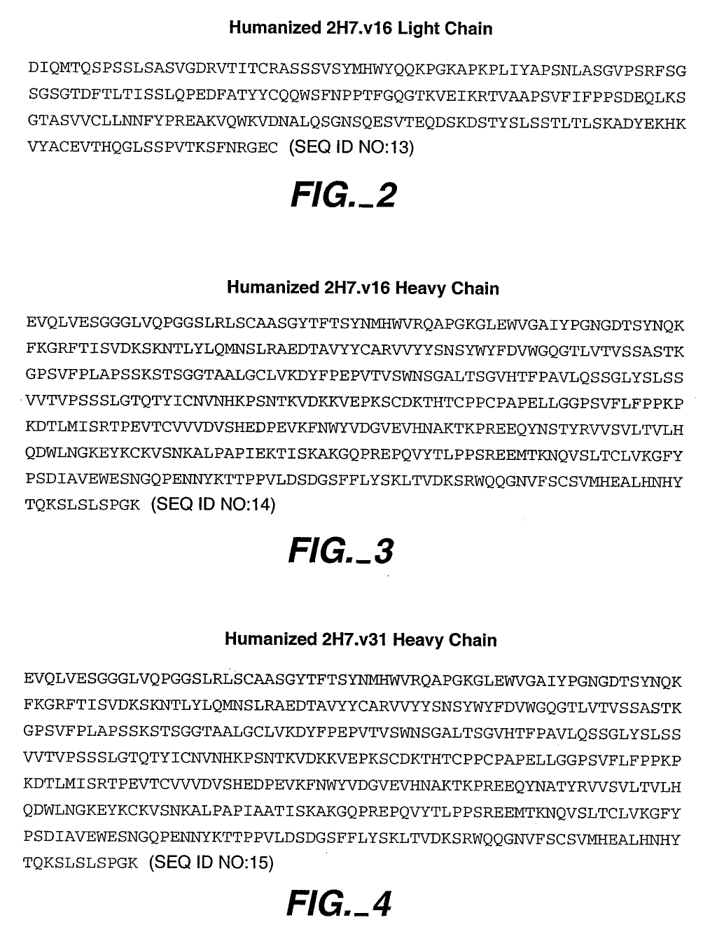

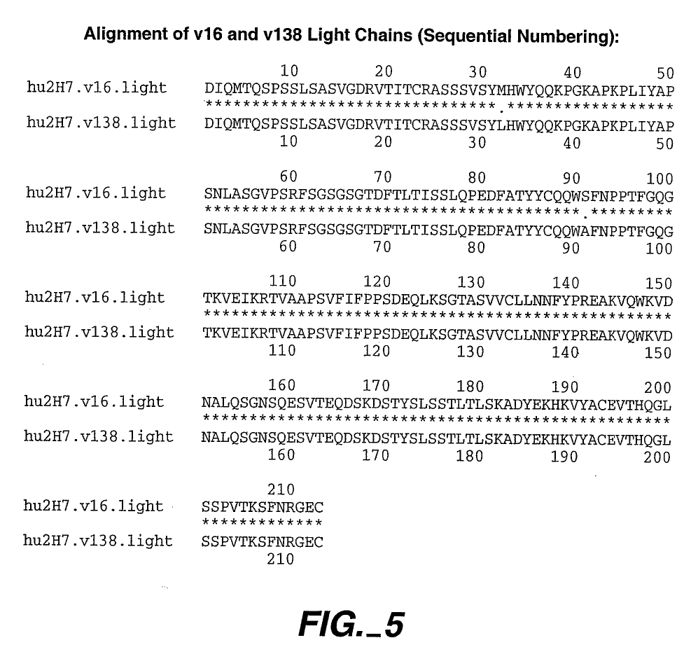

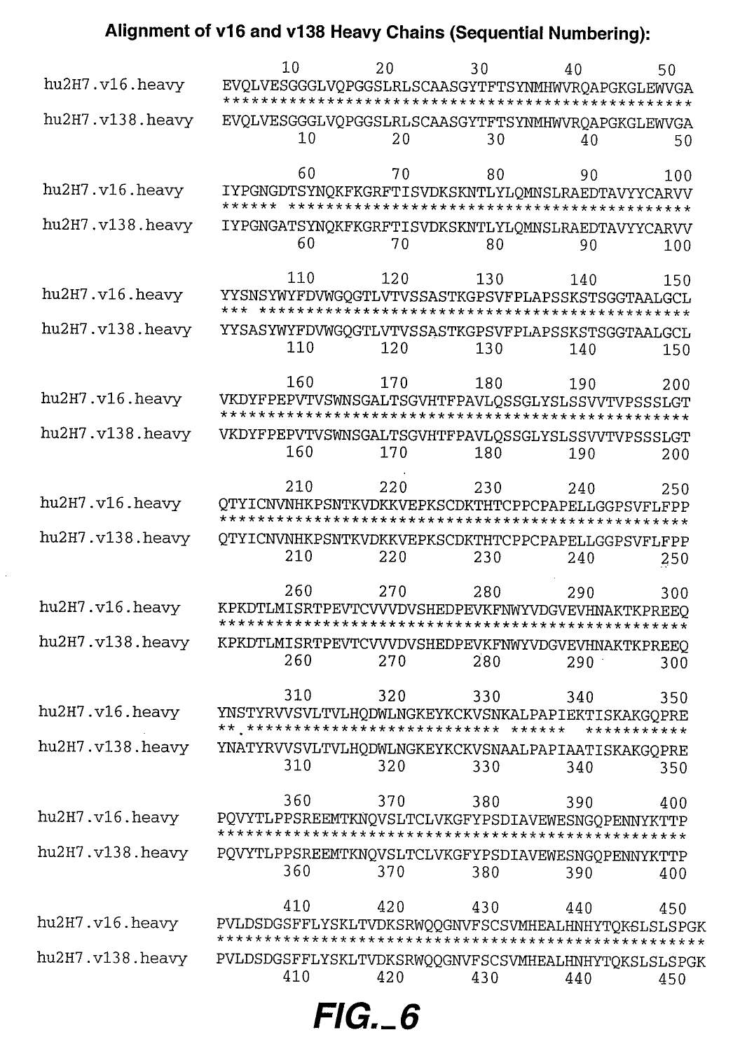

In a preferred embodiment, such antibody comprises the V_{H} sequence of SEQ ID NO:8 (v16, as shown in FIG. 1B), optionally also comprising the V_{L} sequence of SEQ ID NO:2 (v16, as shown in FIG. 1A), which may have the amino acid substitutions of D56A and N100A in the H chain and S92A in the L chain (v96). Preferably, the antibody is an intact antibody comprising the light- and heavy-chain amino acid sequences of SEQ ID NOS:13 and 14, respectively, as shown in FIGS. 2 and 3. Another preferred embodiment is where the antibody is 2H7.v31 comprising the light- and heavy-chain amino acid sequences of SEQ ID NOS:13 and 15, respectively, as shown in FIGS. 2 and 4. The antibody herein may further comprise at least one amino acid substitution in the Fc region that improves ADCC and/or CDC activity, such as one wherein the amino acid substitutions are S298A/E333A/K334A, more preferably 2H7.v31 having the heavy chain amino acid sequence of SEQ ID NO:15 (as shown in FIG. 4). Another preferred embodiment is where the antibody is 2H7.v138 comprising the light- and heavy-chain amino acid sequences of SEQ ID NOS:28 and 29, respectively, as shown in FIGS. 5 and 6, which are alignments of such sequences with the corresponding light- and heavy-chain amino acid sequences of 2H7.v16. Alternatively, such preferred intact humanized 2H7 antibody is 2H7.v477, which has the light- and heavy-chain sequences of 2H7.v138 except for the amino acid substitution of N434W. Any of these antibodies may further comprise at least one amino acid substitution in the Fc region that decreases CDC activity, for example, comprising at least the substitution K322A. See U.S. Pat. No. 6,528,624B1 (Idusogie et al.).

The most preferred humanized 2H7 variants are those having the variable light-chain domain of SEQ ID NO:2 and the variable heavy-chain domain of SEQ ID NO:8, i.e., those with or without substitutions in the Fc region, and those having a variable heavy-chain domain with alteration N100A or D56A and N100A in SEQ ID NO:8 and a variable light-chain domain with alteration M32L, or S92A, or M32L and S92A in SEQ ID NO:2, i.e., those with or without substitutions in the Fc region. If substitutions are made in the Fc region, they are preferably one of those set forth in the table below.

In a summary of various preferred embodiments of the invention, the V region of variants based on 2H7 version 16 will have the amino acid sequences of v16 except at the positions of amino acid substitutions that are indicated in the table below. Unless otherwise indicated, the 2H7 variants will have the same L chain as that of v16.

| 2H7 | Heavy chain | Light chain | |
|---|---|---|---|
| version | (V_{H}) changes | (V_{L}) changes | Fc changes |
| 16 | | | — |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, |
| | | | E356D, M358L |
| 116 | D56A, N100A | M32L, S92A | S298A, K334A, K322A |
| 138 | D56A, N100A | M32L, S92A | S298A, E333A, K334A, |
| | | | K326A |
| 477 | D56A, N100A | M32L, S92A | S298A, E333A, K334A, |
| | | | K326A, N434W |
| 375 | — | — | K334L |

A particularly preferred humanized 2H7 is an intact antibody or antibody fragment comprising the variable light-chain sequence:

| | |
|---|---|
| DIQMTQSPSSLSASVGDRVTITCRASSSVSYMHWYQQKPGKAPKPLIYAPSNLASGVPSRFS | (SEQ ID NO: 2) |
| GSGSGTDFTLTISSLQPEDFATYYCQQWSFNPPTFGQGTKVEIKR; | |

and the variable heavy-chain sequence:

| | |
|---|---|
| EVQLVESGGGLVQPGGSLRLSCAASGYTFTSYNMHWVRQAPGKGLEWVGAIYPGNGDTS | (SEQ ID NO: 8) |
| YNQKFKGRFTISVDKSKNTLYLQMNSLRAEDTAVYYCARVVYYSNSYWYFDVWGQGTL | |
| VTVSS. | |

Where the humanized 2H7 antibody is an intact antibody, preferably it comprises the light-chain amino acid sequence:

| | |
|---|---|
| DIQMTQSPSSLSASVGDRVTITCRASSSVSYMHWYQQKPGKAPKPLIYAPSNLASGVPSRFS | (SEQ ID NO: 13) |
| GSGSGTDFTLTISSLQPEDFATYYCQQWSFNPPTFGQGTKVEIKRTVAAPSVFIFPPSDEQLK | |
| SGTASVVCLLNNFYPREAKVQWKVDNALQSGNSQESVTEQDSKDSTYSLSSTLTLSKADY | |
| EKHKVYACEVTHQGLSSPVTKSFNRGEC; | |

and the heavy-chain amino acid sequence:

| | |
|---|---|
| EVQLVESGGGLVQPGGSLRLSCAASGYTFTSYNMHWVRQAPGKGLEWVGAIYPGNGDTS | (SEQ ID NO: 14) |
| YNQKFKGRFTISVDKSKNTLYLQMNSLRAEDTAVYYCARVVYYSNSYWYFDVWGQGTL | |
| VTVSSASTKGPSVFPLAPSSKSTSGGTAALGCLVKDYFPEPVTVSWNSGALTSGVHTFPAVL | |
| QSSGLYSLSSVVTVPSSSLGTQTYICNVNHKPSNTKVDKKVEPKSCDKTHTCPPCPAPELLG | |
| GPSVFLFPPKPKDTLMISRTPEVTCVVVDVSHEDPEVKENWYVDGVEVHNAKTKPREEQY | |
| NSTYRVVSVLTVLHQDWLNGKEYKCKVSNKALPAPIEKTISKAKGQPREPQVYTLPPSREE | |
| MTKNQVSLTCLVKGFYPSDIAVEWESNGQPENNYKTTPPVLDSDGSFFLYSKLTVDKSRW | |
| QQGNVFSCSVMHEALHNHYTQKSLSLSPGK | |

or the heavy-chain amino acid sequence:

| | |
|---|---|
| EVQLVESGGGLVQPGGSLRLSCAASGYTFTSYNMHWVRQAPGKGLEWVGAIYPGNGDTS | (SEQ ID NO: 15) |
| YNQKFKGRFTISVDKSKNTLYLQMNSLRAEDTAVYYCARVVYYSNSYWYFDVWGQGTL | |
| VTVSSASTKGPSVFPLAPSSKSTSGGTAALGCLVKDYFPEPVTVSWNSGALTSGVHTFPAVL | |
| QSSGLYSLSSVVTVPSSSLGTQTYICNVNHKPSNTKVDKKVEPKSCDKTHTCPPCPAPELLG | |
| GPSVFLFPPKPKDTLMISRTPEVTCVVVDVSHEDPEVKFNWYVDGVEVHNAKTKPREEQY | |
| NATYRVVSVLTVLHQDWLNGKEYKCKVSNKALPAPIAATISKAKGQPREPQVYTLPPSREE | |
| MTKNQVSLTCLVKGFYPSDIAVEWESNGQPENNYKTTPPVLDSDGSFFLYSKLTVDKSRW | |
| QQGNVFSCSVMHEALHNHYTQKSLSLSPGK. | |

In another preferred embodiment, the intact humanized 2H7 antibody comprises the light-chain amino acid sequence:

| | |
|---|---|
| DIQMTQSPSSLSASVGDRVTITCRASSSVSYLHWYQQKPGKAPKPLIYAPSNLASGVPSR | (SEQ ID NO: 28) |
| FSGSGSGTDFTLTISSLQPEDFATYYCQQWAFNPPTFGQGTKVEIKRTVAAPSVFIFPPS | |
| DEQLKSGTASVVCLLNNFYPREAKVQWKVDNALQSGNSQESVTEQDSKDSTYSLSSTLTL | |
| SKADYEKHKVYACEVTHQGLSSPVTKSFNRGEC | |

and the heavy-chain amino acid sequence:

| | |
|---|---|
| EVQLVESGGGLVQPGGSLRLSCAASG | (SEQ ID NO: 29) |
| YTFTSYNMHWVRQAPGKGLEWVGAIYPGNGATSYNQKFKGRFTISVDKSKNTLYLQMNS | |
| L | |
| RAEDTAVYYCARVVYYSASYWYFDVWGQGTLVTVSSASTKGPSVFPLAPSSKSTSGGTAA | |
| LGCLVKDYFPEPVTVSWNSGALTSGVHTFPAVLQSSGLYSLSSVVTVPSSSLGTQTYICN | |
| VNHKPSNTKVDKKVEPKSCDKTHTCPPCPAPELLGGPSVFLFPPKPKDTLMISRTPEVTC | |
| VVVDVSHEDPEVKFNWYVDGVEVHNAKTKPREEQYNATYRVVSVLTVLHQDWLNGKEY | |
| KC | |
| KVSNAALPAPIAATISKAKGQPREPQVYTLPPSREEMTKNQVSLTCLVKGFYPSDIAVEW | |
| ESNGQPENNYKTTPPVLDSDGSFFLYSKLTVDKSRWQQGNVFSCSVMHEALHNHYTQKSL | |
| SLSPGK. | |

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol. 9:457-492 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes that express one or more FcRs and perform effector functions. Preferably, the cells express at least FcγRIII and carry out ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. The preferred FcR is a native-sequence human FcR. Moreover, a preferred FcR is one that binds an IgG antibody (a gamma receptor) and includes receptors of the FcγRI, FcγRIII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-492 (1991); Capel et al., Immunomethods 4:25-34 (1994); and de Haas et al., J. Lab. Clin. Med. 126:330-341 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody that are responsible for antigen binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

A “naked antibody” is an antibody (as herein defined) that is not conjugated to a heterologous molecule, such as a cytotoxic moiety or radiolabel.

An “isolated” antibody is one that has been identified and separated and/or recovered from a component of its natural environment; Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“ANCA-associated vasculitis” or “anti-neutrophil cytoplasmic antibodies-associated vasculitis” or “AAV” as used herein is an autoimmune disease or disorder involving systemic vasculitis (or inflammation of blood-vessel walls) in which circulating anti-neutrophil cytoplasmic antibodies (ANCA) are normally present in the blood of the subject, or other clinical manifestations are present that define the vasculitis, as noted below. The term “ANCA-associated vasculitis” as used herein applies to ANCA-associated vasculitis no matter what the type and stage or severity, and no matter what symptoms are evident, provided the diagnosis is made. Examples of ANCA-associated vasculitis include microscopic polyangiitis, Wegener's granulomatosis, Churg-Strauss syndrome, renal-limited vasculitis (idiopathic necrotizing crescentic glomerulonephritis), and certain types of drug-induced vasculitis. Diagnoses for ANCA-associated vasculitis and its various manifestations include those set forth below.

Several diagnostic tests are commonly used in people suspected of having ANCA-associated vasculitis. Features that may aid in defining the specific type of vasculitic disorder include the type of organ involvement, presence and type of ANCA (myeloperoxidase-ANCA or proteinase 3-ANCA), presence of serum cryoglobulins, and the presence of evidence for granulomatous inflammation.

Exemplary auto-antibodies associated with ANCA-associated vasculitis include elevated level of anti-nuclear antibodies (ANA), anti-rheumatoid factor (RF) antibodies, creatinine, blood urea nitrogen, anti-endothelial antibodies, anti-neutrophil cytoplasmic antibodies (ANCA), such as autoantibodies directed against proteinase 3 (PR3) or against myeloperoxidase (MPO), or a combination thereof.

The ANCA antibodies can be detected using antigen-specific immunochemical assay to characterize PR3-ANCA and MPO-ANCA. Niles et al., supra. Since an ELISA test for ANCA is associated with a substantially higher positive predictive value and likelihood ratios for ANCA-associated vasculitis, ELISA tests may be performed only on samples that are positive for ANCA by immunofluorescence. Stone et al., Arthritis Care and Research, 13: 424-34 (2000); Comment on Arthritis Care Res. 13: 341-342 (2000); Russell et al., Clin. Immunol., 103: 196-203 (2002).

About 10 percent of patients with microscopic polyangiitis (the most common type of ANCA-associated vasculitis) and Wegener's granulomatosis have negative assays for ANCA; however, this finding does not completely rule out these diseases, and ANCA titers do not always correlate with disease activity. Jennette and Falk, N. Engl J. Med., supra. On the other hand, a positive ANCA assay result is not solely diagnostic of ANCA-associated vasculitis.

Table 1 summarizes the potential clinical manifestations of ANCA-associated vasculitis, which should be suspected in any patient who presents with a multisystem disease not caused by an infectious or malignant process (e.g., renal dysfunction, skin rashes, pulmonary manifestations, or neurologic manifestation). Constitutional symptoms are common. The frequency and combination of various system involvements vary among individual disease entities. See also Guillevin et al. Arthritis Rheum. 42:421-430 (1999); Pettersson et al., Clin. Nephrol. 43:141-149 (1995); Savage et al., Lancet 349:553-558 (1997); Guillevin et al., Br. J. Rheumatol 35:958-964 (1996).

TABLE 1
Clinical Manifestations of ANCA-Associated Vasculitis

| System | Manifestations |
|---|---|
| Constitutional | Fever, weight loss, anorexia, general malaise |
| Musculoskeletal | Myalgia, arthralgia |
| Skin | Palpable purpura, urticaria |
| Kidneys | Proteinuria, hematuria, renal insufficiency, |
| | renal failure, necrotizing glomerulonephritis |
| Respiratory tract | Dyspnea, cough, hemoptysis; lung infiltrate, |
| | interstitial lung disease, pulmonary hemorrhage |
| Nervous system | Peripheral neuropathy, especially mononeuritis |
| Gastrointestinal tract | Fecal blood, elevated liver enzymes; diarrhea, |
| | nausea, vomiting, abdominal pain |

The most common cutaneous lesion is palpable purpura—a slightly raised, non-blanching eruption that usually begins in the lower extremities. Occasionally, the rash is vesicular or slightly ulcerated. Urticaria can also be a manifestation of ANCA-associated vasculitis. Unlike non-vasculitic allergic urticaria, vasculitic urticaria lasts more than one day and may evolve into purpuric lesions. The presence of hypocomplementemia may indicate that the vasculitis is immune complex-mediated rather than ANCA-associated vasculitis.

Renal involvement in vasculitis may progress to renal failure. Results of biopsy of the kidney commonly reveal glomerulonephritis. Focal necrosis, crescentic formation and the absence or paucity of immunoglobulin deposits characterize glomerulonephritis in patients with ANCA-associated vasculitis. Pettersson et al., Clin. Nephrol. 43:141-149 (1995). Lung involvement ranges from fleeting focal infiltrates or interstitial disease to massive pulmonary hemorrhagic alveolar capillaritis. The latter is the most life-threatening feature of small-vessel vasculitis.

It is important, however, to differentiate ANCA-associated vasculitis from other diseases that result in multi-system manifestations. Diseases with widespread embolization to different organs (e.g., atheroembolic disease, endocarditis, antiphospholipid syndrome, and atrial myxoma) can produce similar clinical presentations. Kelley, “Vasculitis and related disorders” In: Textbook, of rheumatology. 5th ed. (Philadelphia: Saunders, 1997), pp. 1079-1101. Persons with sepsis can also present with multisystem involvement. It is also important to realize that ANCA-associated vasculitis may be secondary to infections or malignancy. Some viral, bacterial, and fungal infections may be complicated by vasculitis, which is predominantly a dermal vasculitis. The diagnosis is suggested by the clinical history. Malignancy, such as lymphomas, leukemia, myeloproliferative, and myelodysplastic syndromes, may be associated with ANCA-associated vasculitis; however, solid tumors are less commonly associated with such vasculitis. Underlying infectious or malignant causes should be thoroughly evaluated before the diagnosis of ANCA-associated vasculitis is made-even if the ANCA assay result is positive.

Table 2 depicts some of the clinical features that may help in the diagnosis of the specific type of vasculitis. Laboratory assessment should include a complete blood cell count and routine chemistry profile, urinalysis, fecal occult blood test, and chest radiography. There may be normocytic anemia, thrombocytosis, elevated erythrocyte sedimentation rate, increased liver function, or evidence of renal involvement. ANCA serum levels should also be measured. Other laboratory tests that should be performed to exclude ANCA-associated vasculitis include anti-nuclear antibody, rheumatoid factor, cryoglobulins, complement, antibodies to hepatitis B and C, and human immunodeficiency virus (HIV) testing. Chest and sinus computed tomographic scans may also be performed, if appropriate. Pathologic examination of the involved tissue (e.g., skin, nerve, lung, or kidney) may aid in documenting the type of ANCA-associated vasculitis. Biopsy should be obtained from symptomatic and accessible sites. Biopsies from asymptomatic sites have a low yield of positive results.

TABLE 2
Clinical Features That Favor Diagnosis
of a Specific Type of Vasculitis

| Clinical features | Probable type of vasculitis |
|---|---|
| Pulmonary and renal symptoms | Wegener's granulomatosis; |
| | Microscopic polyangiitis |
| Pulmonary-dermal symptoms | Cryoglobulinemia; Henoch- |
| | Schonlein purpura |
| Asthma and eosinophilia | Churg-Strauss syndrome |
| Upper respiratory tract | Wegener's granulomatosis |
| involvement | (e.g., sinusitis and otitis |
| | media) |

Information in Table 2 is from Jennette and Falk, N. Engl. J. Med., supra, and Kelley, supra.

Wegener's granulomatosis is characterized by necrotizing granulomas of the upper and lower respiratory tract together with glomerulonephritis and systemic vasculitis, which involves usually the medium-sized vessels, with formation of granulomas and necrosis of the parenchyma. Kelley, supra. Upper respiratory tract signs and symptoms include sinusitis, nasal ulcers, otitis media, or hearing loss. Upper respiratory tract signs and symptoms are seen in 70 percent of patients and pulmonary infiltrates or nodules that may cavitate develop in 85 percent of patients. Kelley, supra. Serum antiprotease 3-ANCA (c-ANCA) is positive in 75 to 90 percent, although 20 percent may have positive p-ANCA. Open lung biopsy is the most definitive diagnostic test. Sinus biopsy is diagnostic in only 30 percent of cases because inflammatory findings are often nonspecific and renal biopsy is also relatively nonspecific. Radiographic findings are of mid and lower zone opacities, which are diffuse, and both alveolar and interstitial. Nodules, which may cavitate, are rare in children. CT scanning may show diffuse, ill-defined perivascular opacities. Wegener's granulomatosis can affect patients at any age, with the peak incidence during the fourth decade of life and is slightly more common in men. Duna et al., Rheum. Dis. Clin. North Am. 21:949-986 (1995). The most definite way to diagnose Wegener's granulomatosis is by performing a biopsy of an involved organ site (usually the sinuses, lung or kidney) to confirm the presence of vasculitis and granulomas, which together are diagnostic of the disease.

Microscopic polyangiitis is characterized by the presence of ANCA and few or no immune deposits in the involved vessels. Savage et al., Lancet 349:553-558 (1997). The kidneys are the most commonly affected organs in 90 percent of patients who have this type of vasculitis. Kelley, supra. Patients present with variable combinations of renal manifestations, palpable purpura, abdominal pain, cough, and hemoptysis. Most patients have positive MPO-ANCA (p-ANCA), although PR3— ANCA (c-ANCA) may be also present in 40 percent of patients. The most common age of onset is 40 to 60 years and most common sex is men.

Churg-Strauss syndrome is a rare disease and has three phases: allergic rhinitis and asthma, eosinophilic infiltrative disease resembling pneumonia, and systemic small vessel vasculitis with granulomatous inflammation. Guillevin et al., Br. J. Rheumatol., 35:958-964 (1996). The vasculitic phase usually develops within three years of the onset of asthma. Almost all patients have more than 10 percent eosinophils in the blood. Coronary arteritis and myocarditis are the principal causes of morbidity and mortality. The age of onset varies from 15 to 70 years and is more common in men. Drug-induced vasculitis usually develops within seven to 21 days after a drug is started and may be confined to the skin. Jennette and Falk, N. Engl. J. Med., supra. Skin lesions are identical to those seen in systemic small vessel vasculitis. Drugs cause approximately 10 percent of vasculitic skin lesions. Drugs that have been implicated include penicillin, aminopenicillins, sulfonamides, allopurinol, thiazides, quinolones, hydantoins, and propylthiouracil. Some drugs, such as propylthiouracil and hydralazine (APRESOLINE™), appear to cause vasculitis by inducing ANCA.

Another way to test for active disease and determine which patients/subjects are eligible for treatment is to determine the Birmingham Vasculitis Activity Score/Wegener's granulomatosis (BVAS/WG) value of the patient, whether major or minor. This score is an index of vasculitis activity and is designed to document clinical features that are directly due to active Wegener's granulomatosis. It has been found to be a valid and reliable disease-specific indicator for Wegener's granulomatosis. Stone et al., Arthritis & Rheumatism, 44: 912-920 (2001). It can also be used for other ANCA-associated vasculitis diseases. The instrument separates the features that represent new or worse disease activity from those that represent persistent activity. Typically, the patient's BVAS/WG score is 3 or greater (or has been 3 or greater within 28 days of treatment). Each major item on the BVAS/WG evaluation form is scored 3 points. Each minor item is scored 1 point. However, another distinction used is that acute disease, either first presentation or relapse, shows a BVAS/WG of at least 10, whereas persistent disease shows a BVAS/WG of at least 4. Lymphopenia may also be a good marker for Wegener's granulomatosis. Izzedine et al., Nephron 92:466-471 (2002).

A “subject” herein is a human subject, including a patient, eligible for treatment for ANCA-associated vasculitis who is experiencing or has experienced one or more signs, symptoms, or other indicators of ANCA-associated vasculitis, has been diagnosed with ANCA-associated vasculitis, whether, for example, newly diagnosed or previously diagnosed and now experiencing a recurrence or relapse, or is at risk for developing ANCA-associated vasculitis. The subject may have been previously treated with CD20 antibody or not so treated. A subject eligible for treatment of ANCA-associated vasculitis may optionally be identified as one who has been screened, as in the blood, for elevated levels of infiltrating CD20 cells or is screened using an assay to detect auto-antibodies, wherein autoantibody production is assessed qualitatively, and preferably quantitatively.

A “patient” herein is a human subject eligible for treatment for ANCA-associated vasculitis who is experiencing or has experienced one or more signs, symptoms, or other indicators of ANCA-associated vasculitis, whether, for example, newly diagnosed or previously diagnosed and now experiencing a recurrence or relapse. The patient may have been previously treated with CD20 antibody or not so treated. A patient eligible for treatment of ANCA-associated vasculitis may optionally be identified as one who is screened using an assay to detect auto-antibodies, such as those noted above, wherein autoantibody production is assessed qualitatively, and preferably quantitatively.

“Treatment” of a subject herein refers to both therapeutic treatment and prophylactic or preventative measures. Those in need of treatment include those already with ANCA-associated vasculitis as well as those in which the ANCA-associated vasculitis is to be prevented. Hence, the subject may have been diagnosed as having the ANCA-associated vasculitis or may be predisposed or susceptible to the ANCA-associated vasculitis. Treatment of a subject includes treatment of a patient.

“Treatment” of a patient herein refers to therapeutic treatment. Those patients in need of treatment are those diagnosed with ANCA-associated vasculitis.

For purposes herein, a patient or subject is in “remission” if he/she has no symptoms of active ANCA-associated vasculitis disease, such as those detectable by the methods disclosed herein, and has had no recurrence of ANCA titers or rising ANCA titers coinciding with or following reconstitution of B cells, since sustained or recurring ANCA levels have been found to be predictive of relapses in patients in clinical remissions from Wegener's granulomatosis. Boomsma et al., Arthritis Rheum., 43: 2025-2033 (2000). Those who are not in remission include, for example, those experiencing a disease flare after reconstitution of B cells, those suffering organ damage such as kidney damage, or those who are asymptomatic but have had a recurrence of ANCA or an ANCA titer rise coinciding with or following reconstitution of B cells. Such subjects and patients experiencing a return of symptoms, including active disease and/or damage to organs, or exhibiting recurring or rising ANCA titers, are those who have “relapsed” or had a “recurrence.”

A “symptom” of ANCA-associated vasculitis is any morbid phenomenon or departure from the normal in structure, function, or sensation, experienced by the subject or patient and indicative of disease, such as those noted above.

The expression “effective amount” refers to an amount of the antibody or antagonist that is effective for treating ANCA-associated vasculitis.

“Antibody exposure” refers to contact with or exposure to the antibody herein in one or more doses administered over a period of time of about 1 day to about 5 weeks. The doses may be given at one time or at a fixed or at irregular time intervals over this period of exposure, such as, for example, one dose weekly for four weeks or two doses separated by a time interval of about 13-17 days. Initial and later antibody exposures are separated in time from each other as described in detail herein.

An exposure not being administered or provided until a certain time “from the initial exposure” or from any prior exposure means that the time for the second or later exposure is measured from the time any of the doses from the prior exposure were administered, if more than one dose was administered in that exposure. For example, when two doses are administered in an initial exposure, the second exposure is not given until at least about 16-54 weeks as measured from the time the first or the second dose was administered within that prior exposure. Similarly, when three doses are administered, the second exposure may be measured from the time of the first, second, or third dose within the prior exposure. Preferably, “from the initial exposure” or from any prior disclosure is measured from the time of the first dose.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies including anti-interferon-alpha, -beta, or -gamma antibodies, anti-tumor necrosis factor (TNF)-alpha antibodies (infliximab (REMICADE®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodornase; RNA or DNA from the host; FK506; RS-61443;, chlorambucil; deoxyspergualin; rapamycin; T-cell receptor (Cohen et al., U.S. Pat. No. 5,114,721); T-cell receptor fragments (Offner et al., Science, 251: 430-432(1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol., 23:113-5 (2002) and see also definition below); biologic agents that interfere with T cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); and pharmaceutically acceptable salts, acids or derivatives of any of the above.

The term “cytokine” is a generic term for proteins released by one cell population that act on another cell as intercellular mediators. Examples of such cytokines are lymphokines, monokines; interleukins (ILs) such as IL-1, IL-1α, IL-2, IL-3, IL-4, IL-5, IL-6, IL-7, IL-8, IL-9, IL-11, IL-12, IL-15, including PROLEUKIN® rIL-2; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; estradiol; hormone-replacement therapy; androgens such as calusterone, dromostanolone propionate, epitiostanol, mepitiostane, or testolactone; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, gonadotropin-releasing hormone; inhibin; activin; mullerian-inhibiting substance; and thrombopoietin. As used herein, the term hormone includes proteins from natural sources or from recombinant cell culture and biologically active equivalents of the native-sequence hormone, including synthetically produced small-molecule entities and pharmaceutically acceptable derivatives and salts thereof.

The term “growth factor” refers to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony stimulating factors (CSFs) such as macrophage-CSF (M-CSF); granulocyte-macrophage-CSF (GM-CSF); and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

The term “integrin” refers to a receptor protein that allows cells both to bind to and to respond to the extracellular matrix and is involved in a variety of cellular functions such as wound healing, cell differentiation, homing of tumor cells and apoptosis. They are part of a large family of cell adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include Alpha6beta1, Alpha3beta1, Alpha7beta1, LFA-1 etc. As used herein, the term “integrin” includes proteins from natural sources or from recombinant cell culture and biologically active equivalents of the native-sequence integrin, including synthetically produced small-molecule entities and pharmaceutically acceptable derivatives and salts thereof.

For the purposes herein, “tumor necrosis factor alpha (TNF-alpha)” refers to a human TNF-alpha molecule comprising the amino acid sequence as described in Pennica et al., Nature, 312:721 (1984) or Aggarwal et al., JBC, 260:2345 (1985). A “TNF-alpha inhibitor” herein is an agent that inhibits, to some extent, a biological function of TNF-alpha, generally through binding to TNF-alpha and neutralizing its activity. Examples of TNF inhibitors specifically contemplated herein are etanercept (ENBREL®), infliximab (REMICADE®), and adalimumab (HUMIRA™).

Examples of “disease-modifying anti-rheumatic drugs” or “DMARDs” include hydroxycloroquine, sulfasalazine, methotrexate, leflunomide, etanercept, infliximab (plus oral and subcutaneous methotrexate), azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197, (9), p1125-39 (2003)), including salts and derivatives thereof, etc.

Examples of “non-steroidal anti-inflammatory drugs” or “NSAIDs” include aspirin, acetylsalicylic acid, ibuprofen, naproxen, indomethacin, sulindac, tolmetin, COX-2 inhibitors such as celecoxib (CELEBREX®; 4-(5-(4-methylphenyl)-3-(trifluoromethyl)-1H-pyrazol-1-yl) benzenesulfonamide and valdecoxib (BEXTRA®), and meloxicam (MOBIC®), including salts and derivatives thereof, etc. Preferably, they are aspirin, naproxen, ibuprofen, indomethacin, or tolmetin.

Examples of “integrin antagonists or antibodies” herein include an LFA-1 antibody, such as efalizumab (RAPTIVA®) commercially available from Genentech, or an alpha 4 integrin antibody such as natalizumab (ANTEGREN®) available from Biogen, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US2002/0042368), antibodies to alphavbeta3 integrin (EP 633945), aza-bridged bicyclic amino acid derivatives (WO 2002/02556), etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

The terms “BAFF,” “BAFF polypeptide,” “TALL-1” or “TALL-1 polypeptide,” and “BLyS” when used herein encompass “native-sequence BAFF polypeptides” and “BAFF variants”. “BAFF” is a designation given to those polypeptides that have any one of the amino acid sequences shown below:

Human BAFF sequence (SEQ ID NO:16):

| 1 | MDDSTEREQSRLTSCLKKREEMKLKECVSILPRKESPSVRSSKDGKLLAATLLLALLSCC |
|---|---|
| 61 | LTVVSFYQVAALQGDLASLRAELQGHHAEKLPAGAGAPKAGLEEAPAVTAGLKIFEPPAP |
| 121 | GEGNSSQNSRNKRAVQGPEETVTQDCLQLIADSETPTIQKGSYTFVPWLLSFKRGSALEE |
| 181 | KENKILVKETGYFFIYGQVLYTDKTYAMGHLIQRKKVHVFGDELSLVTLFRCIQNMPETL |
| 241 | PNNSCYSAGIAKLEEGDELQLAIPRENAQISLDGDVTFFGALKLL |
| Mouse BAFF sequence (SEQ ID NO: 17): | |
| 1 | MDESAKTLPPPCLCFCSEKGEDMKVGYDPITPQKEEGAWFGICRDGRLLAATLLLALLSS |
| 61 | SFTAMSLYQLAALQADLMNLRMELQSYRGSATPAAAGAPELTAGVKLLTPAAPRPHNSSR |
| 121 | GHRNRRAEQGPEETEQDVDLSAPPAPCLPGCRHSQHDDNGMNLRNIIQDCLQLIADSDTP |
| 181 | TIRKGTYTFVPWLLSFKRGNALEEKENKIVVRQTGYFFIYSQVLYTDPIFAMGHVIQRKK |
| 241 | VHVFGDELSLVTLFRCIQNMPKTLPNNSCYSAGIARLEEGDEIQLAIPRENAQISRNGDD |
| 301 | TFFGALKLL |

and homologs and fragments and variants thereof, which have the biological activity of the native BAFF. A biological activity of BAFF can be selected from the group consisting of promoting B cell survival, promoting B cell maturation and binding to BR3. Variants of BAFF will preferably have at least 80% or any successive integer up to 100% including, more preferably, at least 90%, and even more preferably, at least 95% amino acid sequence identity with a native sequence of a BAFF polypeptide.

A “native-sequence” BAFF polypeptide comprises a polypeptide having the same amino acid sequence as the corresponding BAFF polypeptide derived from nature. For example, BAFF exists in a soluble form following cleavage from the cell surface by furin-type proteases. Such native-sequence BAFF polypeptides can be isolated from nature or can be produced by recombinant and/or synthetic means.

The term “native-sequence BAFF polypeptide” or “native BAFF” specifically encompasses naturally occurring truncated or secreted forms (e.g., an extracellular domain sequence), naturally occurring variant forms (e.g., alternatively spliced forms), and naturally occurring allelic variants of the polypeptide. The term “BAFF” includes those polypeptides described in Shu et al., J. Leukocyte Biol., 65:680 (1999); GenBank Accession No. AF136293; WO 1998/18921 published May 7, 1998; EP 869,180 published Oct. 7, 1998; WO 1998/27114 published Jun. 25, 1998; WO 1999/12964 published Mar. 18, 1999; WO 1999/33980 published Jul. 8, 1999; Moore et al., Science, 285:260-263 (1999); Schneider et al., J. Exp. Med., 189:1747-1756 (1999) and Mukhopadhyay et al., J. Biol. Chem., 274:15978-15981 (1999).

The term “BAFF antagonist” as used herein is used in the broadest sense, and includes any molecule that (1) binds a native-sequence BAFF polypeptide or binds a native-sequence of BR3 to partially or fully block BR3 interaction with BAFF polypeptide, and (2) partially or fully blocks, inhibits, or neutralizes native-sequence BAFF activity. In one preferred embodiment the BAFF receptor to be blocked is the BR3 receptor. Native BAFF activity promotes, among other things, B-cell survival and/or B-cell maturation. In one embodiment, the inhibition, blockage or neutralization of BAFF activity results in a reduction in the number of B cells. A BAFF antagonist according to this invention will partially or fully block, inhibit, or neutralize one or more biological activities of a BAFF polypeptide, in vitro and/or in vivo. In one embodiment, a biologically active BAFF potentiates any one or a combination of the following events in vitro and/or in vivo: an increased survival of B cells, an increased level of IgG and/or IgM, an increased numbers of plasma cells, and processing of NF-κb2/100 to p52 NF-κb in splenic B cells (e.g., Batten et al., J. Exp. Med. 192:1453-1465 (2000); Moore et al., Science 285:260-263 (1999); Kayagaki et al. Immunity 17:515-524 (2002)).

As mentioned above, a BAFF antagonist can function in a direct or indirect manner to partially or fully block, inhibit or neutralize BAFF signaling, in vitro or in vivo. For instance, the BAFF antagonist can directly bind BAFF. For example, BAFF antibodies that bind within a region of human BAFF comprising residues 162-275 and/or a neighboring residue of a residue selected from the group consisting of 162, 163, 206, 211, 231, 233, 264 and 265 of human BAFF such that the antibody sterically hinders BAFF binding to BR3 are contemplated, where such residue numbers refer to SEQ ID NO:16. In another example, a direct binder is a polypeptide comprising any portion of a BAFF receptor that binds BAFF such as an extracellular domain of a BAFF receptor, or fragments and variants thereof that bind native BAFF. In another example, BAFF antagonists include the polypeptides having a sequence of a polypeptide comprising the sequence of Formula I:
X₁-C-X₃-D-X₅-L-X₇-X₈-X₉-X₁₀-X₁₁-X₁₂-C-X₁₄-X₁₅-X₁₆-X₁₇ (Formula I) (SEQ ID NO: 18)
wherein X₁, X₃, X₅, X₇, X₈, X₉, X₁₀, X₁₁, X₁₂, X₁₄, X₁₅ and X₁₇ are any amino acid except cysteine; and
wherein X₁₆ is an amino acid selected from the group consisting of L, F, I and V; and
wherein the polypeptide does not comprise a cysteine within seven amino acid residues N-terminal to the most N-terminal cysteine C and C-terminal to the most C-terminal cysteine C of Formula I.

In one embodiment, a polypeptide comprising the sequence of Formula I has the two Cs joined by disulfide bonding; X₅LX₇X₈ forming the conformation of a type 1 beta turn structure with the center of the turn between L and X₇; and has a positive value for the dihedral angle phi of X₈. In one embodiment, X₁₀ is selected from the group consisting of W, F, V, L, I, Y, M and a non-polar amino amino acid. In another embodiment, X₁₀ is W. In another embodiment, X₃ is an amino acid selected from the group consisting of M, V, L, I, Y, F, W and a non-polar amino acid. In another embodiment, X₅ is selected from the group consisting of V, L, P, S, I, A and R. In another embodiment, X₇ is selected from the group consisting of V, T, I and L. In another embodiment, X₈ is selected from the group consisting of R, K, G, N, H and a D-amino acid. In another embodiment, X₉ is selected from the group consisting of H, K, A, R and Q. In another embodiment, X₁₁ is I or V. In another embodiment, X₁₂ is selected from the group consisting of P, A, D, E and S. In another embodiment, X₁₆ is L. In one specific embodiment, the sequence of Formula I is a sequence selected from the group consisting of ECFDLLVRAWVPCSVLK (SEQ ID NO:19), ECFDLLVRHWVPCGLLR (SEQ ID NO:20), ECFDLLVRRWVPCEMLG (SEQ ID NO:21), ECFDLLVRSWVPCHMLR (SEQ ID NO:22), ECFDLLVRHWVACGLLR (SEQ ID NO:23), and QCFDRLNAWVPCSVLK (SEQ ID NO:24). In a preferred embodiment, the BAFF antagonist comprises any one of the amino acid sequences selected from the group consisting of SEQ ID NO: 19, 20, 21, 22, and 23.

In still another example, BAFF antagonists include the polypeptides having a sequence of a polypeptide comprising the sequence of Formula II:
X₁C-X₃-D-X₅-L-V-X₈-X₉-W-V-P-C-X₁₄-X₁₅-L-X₁₇ (Formula II) (SEQ ID NO:25)
wherein X₁, X₃, X₅, X₈, X₉, X₁₄, X₁₅ and X₁₇ are any amino acid, except cysteine; and wherein the polypeptide does not comprise a cysteine within seven amino acid residues N-terminal to the most N-terminal cysteine C and C-terminal to the most C-terminal cysteine C of Formula II.

In one embodiment, a polypeptide comprising the sequence of Formula II has a disulfide bond between the two Cs and has the conformation of X₅LX₇X₈ forming a type 1 beta turn structure with the center of the turn between L and X₇; and has a positive value for the dihedral angle phi of X₈. In another embodiment of Formula II, X₃ is an amino acid selected from the group consisting of M, A, V, L, I, Y, F, W and a non-polar amino acid. In another embodiment of Formula II, X₅ is selected from the group consisting of V, L, P, S, I, A and R. In another embodiment of Formula II, X₈ is selected from the group consisting of R, K, G, N, H and D-amino acid. In another embodiment of Formula II, X₉ is selected from the group consisting of H, K, A, R and Q.

In a further embodiment, the BAFF receptor from which the extracellular domain or BAFF-binding fragment or BAFF-binding variant thereof is derived is TAC1, BR3 or BCMA. Alternatively, the BAFF antagonist can bind an extracellular domain of a native-sequence BR3 at its BAFF binding region to partially or fully block, inhibit or neutralize BAFF binding to BR3 in vitro, in situ, or in vivo. For example, such indirect antagonist is an anti-BR3 antibody that binds in a region of BR3 comprising residues 23-38 of human BR3 as defined below (SEQ ID NO:26) or a neighboring region of those residues such that binding of human BR3 to BAFF is sterically hindered.

In some embodiments, a BAFF antagonist according to this invention includes BAFF antibodies and immunoadhesins comprising an extracellular domain of a BAFF receptor, or fragments and variants thereof that bind native BAFF. In a further embodiment, the BAFF receptor from which the extracellular domain or BAFF-binding fragment or BAFF-binding variant thereof is derived is TAC1, BR3 or BCMA. In a still another embodiment, the immunoadhesin comprises an amino acid sequence of that of Formula I or Formula II as set forth above, including an amino acid sequence selected from any one of the group consisting of SEQ ID NOS: 19, 20, 21, 22, 23, and 24.

According to one embodiment, the BAFF antagonist binds to a BAFF polypeptide or a BR3 polypeptide with a binding affinity of 100 nM or less. According to another embodiment, the BAFF antagonist binds to a BAFF polypeptide or a BR3 polypeptide with a binding affinity of 10 nM or less. According to yet another embodiment, the BAFF antagonist binds to a BAFF polypeptide or a BR3 polypeptide with a binding affinity of 1 nM or less.

The terms “BR3”, “BR3 polypeptide” or “BR3 receptor” when used herein encompass “native-sequence BR3 polypeptides” and “BR3 variants” (which are further defined herein). “BR3” is a designation given to those polypeptides comprising the following amino acid sequence and homologs thereof, and variants or fragments thereof that bind native BAFF: Human BR3 sequence (SEQ ID NO:26):

| | |
|---|---|
| 1 | MRRGPRSLRGRDAPAPTPCVPAECFDLLVRHCVACGLLRTPRPKPAGASSPAPRTALQPQ |
| 61 | ESVGAGAGEAALPLPGLLFGAPALLGLALVLALVLVGLVSWRRRQRRLRGASSAEAPDGD |
| 121 | KDAPEPLDKVIILSPGISDATAPAWPPPGEDPGTTPPGHSVPVPATELGSTELVTTKTAG |
| 181 | PEQQ. |

The BR3 polypeptides of the invention can be isolated from a variety of sources, such as from human tissue types or from another source, or prepared by recombinant and/or synthetic methods. The term BR3 includes the BR3 polypeptides described in WO 2002/24909 and WO 2003/14294.

A “native-sequence” BR3 polypeptide or “native BR3” comprises a polypeptide having the same amino acid sequence as the corresponding BR3 polypeptide derived from nature. Such native-sequence BR3 polypeptides can be isolated from nature or can be produced by recombinant and/or synthetic means. The term “native-sequence BR3 polypeptide” specifically encompasses naturally occurring truncated, soluble or secreted forms (e.g., an extracellular domain sequence), naturally occurring variant forms (e.g., alternatively spliced forms) and naturally occurring allelic variants of the polypeptide. The BR3 polypeptides of the invention include the BR3 polypeptide comprising or consisting of the contiguous sequence of amino acid residues 1 to 184 of a human BR3 (SEQ ID NO:26).

A BR3 “extracellular domain” or “ECD” refers to a form of the BR3 polypeptide that is essentially free of the transmembrane and cytoplasmic domains. ECD forms of BR3 include a polypeptide comprising any one of the amino acid sequences selected from the group consisting of amino acids 1-77, 2-62, 2-71, 1-61, 7-71, 23-38 and 2-63 of human BR3. The invention contemplates BAFF antagonists that are polypeptides comprising any one of the above-mentioned ECD forms of human BR3 and variants and fragments thereof that bind a native BAFF.

Mini-BR3 is a 26-residue core region of the BAFF-binding domain of BR3, i.e., the amino acid sequence: TPCVPAECFD LLVRHCVACG LLRTPR (SEQ ID NO:27)

“BR3 variant” means a BR3 polypeptide having at least about 80% amino acid sequence identity with the amino acid sequence of a native-sequence, full-length BR3 or BR3 ECD and binds a native-sequence BAFF polypeptide. Optionally, the BR3 variant includes a single cysteine-rich domain. Such BR3 variant polypeptides include, for instance, BR3 polypeptides wherein one or more amino acid residues are added, or deleted, at the N- and/or C-terminus, as well as within one or more internal domains, of the full-length amino acid sequence. Fragments of the BR3 ECD that bind a native sequence BAFF polypeptide are also contemplated. According to one embodiment, a BR3 variant polypeptide will have at least about 80% amino acid sequence identity, at least about 81% amino acid sequence identity, at least about 82% amino acid sequence identity, at least about 83% amino acid sequence identity, at least about 84% amino acid sequence identity, at least about 85% amino acid sequence identity, at least about 86% amino acid sequence identity, at least about 87% amino acid sequence identity, at least about 88% amino acid sequence identity, at least about 89% amino acid sequence identity, at least about 90% amino acid sequence identity, at least about 91% amino acid sequence identity, at least about 92% amino acid sequence identity, at least about 93% amino acid sequence identity, at least about 94% amino acid sequence identity, at least about 95% amino acid sequence identity, at least about 96% amino acid sequence identity, at least about 97% amino acid sequence identity, at least about 98% amino acid sequence identity or at least about 99% amino acid sequence identity with a human BR3 polypeptide or a specified fragment thereof (e.g., ECD). BR3 variant polypeptides do not encompass the native BR3 polypeptide sequence. According to another embodiment, BR3 variant polypeptides are at least about 10 amino acids in length, at least about 20 amino acids in length, at least about 30 amino acids in length, at least about 40 amino acids in length, at least about 50 amino acids in length, at least about 60 amino acids in length, or at least about 70 amino acids in length.

In one preferred embodiment, the BAFF antagonists herein are immunoadhesins comprising a portion of BR3, TACI or BCMA that binds BAFF, or variants thereof that bind BAFF. In other embodiments, the BAFF antagonist is a BAFF antibody. A “BAFF antibody” is an antibody that binds BAFF, and preferably binds BAFF within a region of human BAFF comprising residues 162-275 of the human BAFF sequence disclosed herein under the “BAFF” definition (SEQ ID NO:16). In another embodiment, the BAFF antagonist is BR3 antibody. A “BR3 antibody” is an antibody that binds BR3, and is preferably one that binds BR3 within a region of human BR3 comprising residues 23-38 of the human BR3 sequence disclosed herein under the “BR3” definition (SEQ ID NO:26). In general, the amino acid positions of human BAFF and human BR3 referred to herein are according to the sequence numbering under human BAFF and human BR3, SEQ ID NOS: 16 and 26, respectively, disclosed herein under the “BAFF” and “BR3” definitions.

Other examples of BAFF-binding polypeptides or BAFF antibodies can be found in, e.g., WO 2002/092620, WO 2003/014294, Gordon et al., Biochemistry 42(20):5977-5983 (2003), Kelley et al., J. Biol. Chem.,279(16):16727-16735 (2004), WO 1998/18921, WO 2001/12812, WO 2000/68378 and WO 2000/40716.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

A “medicament” is an active drug to treat the ANCA-associated vasculitis or its symptoms or side effects.

II. Therapy

In one aspect, the present invention provides a method of treating ANCA-associated vasculitis in a patient comprising administering an antagonist, preferably an antibody, that binds to a B-cell surface marker (more preferably a CD20 antibody) to the patient in a dose of about 400 mg to 1.3 grams at a frequency of one to three doses within a period of about one month.

Thus, the invention contemplates a method of treating ANCA-associated vasculitis in a patient comprising administering an antibody that binds to a B-cell surface marker to the patient in a dose of about 400 mg to 1.3 grams at a frequency of one to three doses within a period of about one month.

The invention also contemplates a method of treating ANCA-associated vasculitis in a patient comprising administering an antagonist that binds to a B-cell surface marker to the patient in a dose of about 400 mg to 1.3 grams at a frequency of one to three doses within a period of about one month.

The invention also contemplates a method of treating ANCA-associated vasculitis in a patient comprising administering a CD20 antibody to the patient in a dose of about 400 mg to 1.3 grams at a frequency of one to three doses within a period of about one month.

In a preferred embodiment of each of these aspects, the dose is about 500 mg to 1.2 grams, more preferably about 750 mg to 1.1 grams. In another preferred embodiment, the antibody is administered in two to three doses, more preferably in two doses, but alternatively three doses. In a still preferred embodiment, the antibody is administered within a period of about 2 to 3 weeks, more preferably about two weeks, but alternatively three weeks.

In another embodiment, the present invention provides a method of treating ANCA-associated vasculitis in a subject eligible for treatment, comprising administering an effective amount of an antibody that binds to a B-cell surface marker (preferably a CD20 antibody) to the subject to provide an initial antibody exposure (of preferably about 0.5 to 4 grams, more preferably about 1.5 to 3.5 grams, and still more preferably about 1.5 to 2.5 grams) followed by a second antibody exposure (of preferably about 0.5 to 4 grams, more preferably about 1.5 to 3.5 grams, still more preferably about 1.5 to 2.5 grams), the second exposure not being provided until from about 16 to 54 weeks (preferably from about 20 to 30 weeks, more preferably from about 46 to 54 weeks) from the initial exposure.

For purposes of this invention, the second antibody exposure is the next time the subject is treated with the CD20 antibody after the initial antibody exposure, there being no intervening CD20 antibody treatment or exposure between the initial and second exposures. Such re-treatment may be scheduled or unscheduled, but is preferably a scheduled redosing, particularly to protect organs such as kidneys from damage.

The method preferably comprises administering to the subject an effective amount of the CD20 antibody to provide a third antibody exposure (preferably of about 0.5 to 4 grams, more preferably about 1.5 to 3.5 grams, still more preferably about 1.5 to 2.5 grams), the third exposure not being provided until from about 46 to 60 weeks (preferably about 46 to 55, more preferably about 46 to 52 weeks) from the initial exposure. Preferably, no further antibody exposure is provided until at least about 70-75 weeks from the initial exposure, and still more preferably no further antibody exposure is provided until about 74 to 80 weeks from the initial exposure.

Any one or more of the antibody exposures herein may be provided to the subject as a single dose of antibody, or as separate doses, for example, about 1-4 separate doses of the antibody (e.g., constituting a first and second dose, or a first, second, and third dose, or a first, second, third, and fourth dose, etc). The particular number of doses (whether one, two or three or more) employed for each antibody exposure is dependent, for example, on the type of ANCA-associated vasculitis treated, the type of antibody employed, whether, what type, and how much and how many of a second medicament is employed as noted below, and the method and frequency of administration. Where separate doses are administered, the later dose (for example, second or third dose) is preferably administered from about 1 to 20 days, more preferably from about 6 to 16 days, and most preferably from about 14 to 16 days from the time the previous dose was administered. The separate doses are preferably administered within a total period of between about 1 day and 4 weeks, more preferably between about 1 and 20 days (e.g., within a period of 6-18 days). In one such aspect, the separate doses are administered about weekly, with the second dose being administered about one week from the first dose and any third or subsequent dose being administered about one week from the second dose. Each such separate dose of the antibody is preferably about 0.5 to 1.5 grams, more preferably about 0.75 to 1.3 grams.

In a most preferred embodiment, a method of treating ANCA-associated vasculitis in a subject is provided comprising administering an effective amount of an antibody that binds to a B-cell surface marker (e.g., a CD20 antibody) to the subject to provide an initial antibody exposure followed by a second antibody exposure, wherein the second exposure is not provided until from about 16 to 54 weeks from the initial exposure and each of the antibody exposures is provided to the subject as a single dose or as two or three separate doses of antibody. Preferably in such a method, the antibody exposures are of about 0.5 to 4 grams each, and most preferably the amounts given above.

In one embodiment, the subject is provided at least about three exposures of the antibody, for example, from about 3 to 60 exposures, and more particularly about 3 to 40 exposures, most particularly, about 3 to 20 exposures. Preferably, such exposures are administered at intervals each of 24 weeks. In one embodiment, each antibody exposure is provided as a single dose of the antibody. In an alternative embodiment, each antibody exposure is provided as separate doses of the antibody. However, not every antibody exposure need be provided as a single dose or as separate doses.

In one preferred embodiment, about 2-3 grams of the CD20 antibody is administered as the initial exposure. If about 3 grams are administered, then about 1 gram of the CD20 antibody is administered weekly for about three weeks as the initial exposure. If about 2 grams of the CD20 antibody is administered as the initial exposure, then about 1 gram of the CD20 antibody is administered followed in about two weeks by another about 1 gram of the antibody as the initial exposure. In a preferred aspect, the second exposure is at about six months from the initial exposure and is administered in an amount of about 2 grams. In an alternative preferred aspect, the second exposure is at about six months from the initial exposure and is administered as about 1 gram of the antibody followed in about two weeks by another about 1 gram of the antibody.

In all the inventive methods set forth herein, the CD20 or B-cell surface marker antibody may be a naked antibody or may be conjugated with another molecule such as a cytotoxic agent such as a radioactive compound. The preferred CD20 antibody herein is a chimeric, humanized, or human CD20 antibody, more preferably rituximab, a humanized 2H7 (e.g. comprising the variable domain sequences in SEQ ID Nos. 2 and 8, or comprising a variable heavy-chain domain with alteration N100A or D56A and N100A in SEQ ID NO:8 and a variable light-chain domain with alteration M32L, or S92A, or M32L and S92A in SEQ ID NO:2), chimeric or humanized A20 antibody (Immunomedics), or HUMAX-CD20™ human CD20 antibody (Genmab). Still more preferred is rituximab or a humanized 2H7. Also, while the ANCA-associated vasculitis in all methods herein can be any such disease, in one preferred embodiment, it is Wegener's granulomatosis or microscopic polyangiitis.

In a further embodiment of all the methods herein, the subject or patient has never been previously treated with drug(s), such as immunosuppressive agent(s), to treat the ANCA-associated vasculitis and/or has never been previously treated with an antagonist (for example, antibody) to a B-cell surface marker (e.g. never been previously treated with a CD20 antibody). In a still further aspect, the subject or patient may have had a relapse with the ANCA-associated vasculitis or suffered organ damage such as kidney damage before being treated in any of the methods above, including after the initial or a later antibody exposure. However, preferably, the patient or subject has not relapsed with the vasculitis and more preferably has not had such a relapse before at least the initial treatment.

In another embodiment, the subject or patient has been previously treated with drug(s) to treat the vasculitis and/or has been previously treated with such antibody or antagonist. In another embodiment, the antagonist (for example, CD20 antibody) is the only medicament administered to the subject or patient to treat the vasculitis. In another embodiment, the antagonist (e.g., CD20 antibody) is one of the medicaments used to treat the vasculitis. In a further embodiment, the subject or patient does not have a malignancy. In a still further embodiment, the subject or patient does not have rheumatoid arthritis. In a still further embodiment, the subject or patient does not have multiple sclerosis. In a yet further embodiment, the subject or patient does not have lupus or Sjögren's syndrome. In yet another embodiment, the subject or patient does not have an autoimmune disease other than ANCA-associated vasculitis. In yet another aspect of the invention, the ANCA-associated vasculitis is not associated with a different autoimmune disease or with a risk of developing a different autoimmune disease. For purposes of these lattermost statements, an “autoimmune disease” herein is a disease or disorder arising from and directed against an individual's own tissues or organs or a co-segregate or manifestation thereof or resulting condition therefrom. Without being limited to any one theory, B cells demonstrate a pathogenic effect in human autoimmune diseases through a multitude of mechanistic pathways, including autoantibody production, immune complex formation, dendritic and T-cell activation, cytokine synthesis, direct chemokine release, and providing a nidus for ectopic neo-lymphogenesis. Each of these pathways participates to different degrees in the pathology of autoimmune diseases.

In still another embodiment, the subject or patient has a BVAS/WG score of less than 3, more preferably less than about 2, still more preferably less than about 1, and most preferably 0 (complete remission) at about three months, preferably about six months, and most preferably about one year or greater, after administration of the antagonist or antibody. Specific embodiments of this BVAS response are achieving a BVAS/WG score of less than 2 at three months after administration, or less than 1 (for example, 0.2 or 0.4) at 14 weeks or three months after administration, or less than 1 (for example, 0.6) at 6 months after administration, or most preferably, 0 at three or six months after administration. In another embodiment, the amount of steroid such as prednisone as compared to start of treatment is lessened without substantially affecting the lowered BVAS/WG score. Thus, for example, a subject or patient at a set interval after treatment (such as three months or six months after treatment) preferably has a lowered BVAS/WG score from baseline and is being administered less of a dose of a steroid from baseline (baseline being at the start of administration). In a still further embodiment, a step is included in the treatment method to test for the subject's or patient's response to treatment after the administration step to determine that the level of response is effective to treat the vasculitis. For example, a step is included to test the BVAS/WG score after administration and compare it to a baseline BVAS/WG score obtained before administration to determine if treatment is effective by measuring if, and by how much, it has been lowered. This test may be repeated at various scheduled or unscheduled time intervals after the administration to determine maintenance of any partial or complete remission. Alternatively, the methods herein comprise a step of testing the patient or subject, before administration, to see if one or more biomarkers are present for ANCA-associated vasculitis, such as one or more autoantibodies, a BVAS/WG score, or symptoms unique to ANCA-associated vasculitis, as set forth above. In another method, a step may be included to check the patient's or subject's clinical history, as detailed above, for example, to rule out infections or malignancy as causes, for example, primary causes, of the patient's or subject's condition, prior to administering the antibody or antagonist to the subject or patient. Preferably, the ANCA-associated vasculitis is primary (i.e., the leading disease), and is not secondary, such as secondary to infection or malignancy, whether solid or liquid tumors.

In a preferred embodiment of the multi-exposure method herein, the subject is in remission after the initial or any later antibody exposures. More preferably, the multi-exposure method herein involves scheduled re-dosing or re-treating such that the patient is in remission when provided the second, and preferably all antibody exposures. Such re-dosing is scheduled to prevent any relapse, recurrence, or organ damage, rather than to treat it therapeutically. Most preferably, the subject is in remission for at least about six months, and still most preferably at least about nine months, and even still most preferably at least about a year since the last antibody exposure used in the re-treatment method.

In yet another embodiment, the subject is treated with the same CD20 antibody for at least two antibody exposures, and preferably for each antibody exposure. Thus, the initial and second antibody exposures are preferably with the same antibody, and more preferably all antibody exposures are with the same antibody, i.e., treatment for the first two exposures, and preferably all exposures, is with one type of antibody that binds to a B-cell surface marker, such as CD20 antibody, e.g., all with rituximab or all with the same humanized 2H7.

In any of the methods herein, one may administer to the subject or patient along with the antagonist or antibody that binds a B-cell surface marker an effective amount of a second medicament (where the antagonist or antibody that binds a B-cell surface marker (e.g., the CD20 antibody) is a first medicament). The second medicament may be one or more medicaments, and include, for example, a cytotoxic agent, chemotherapeutic agent, immunosuppressive agent, cytokine, cytokine antagonist or antibody, growth factor, hormone, integrin, integrin antagonist or antibody, or any combination thereof. The type of such second medicament depends on various factors, including the type of vasculitis, the severity of the vasculitis, the condition and age of the patient, the type and dose of first medicament employed, etc.

Examples of such additional medicaments include a chemotherapeutic agent, an interferon class drug such as interferon-alpha (e.g., from Amarillo Biosciences, Inc.), IFN-beta-1a (REBIF® and AVONEX®) or IFN-beta-1b (BETASERON®), an oligopeptide such as glatiramer acetate (COPAXONE®), an agent blocking CD40-CD40 ligand, a cytotoxic or immunosuppressive agent (such as mitoxantrone (NOVANTRONE®), methotrexate, cyclophosphamide, chlorambucil, leflunomide, and azathioprine), intravenous immunoglobulin (gamma globulin), lymphocyte-depleting therapy (e.g., mitoxantrone, cyclophosphamide, CAMPATH™ antibodies, anti-CD4, cladribine, a polypeptide construct with at least two domains comprising a de-immunized, autoreactive antigen or its fragment that is specifically recognized by the Ig receptors of autoreactive B-cells (WO 2003/68822), total body irradiation, bone marrow transplantation), integrin antagonist or antibody (e.g., an LFA-1 antibody such as efalizumab/RAPTIVA® commercially available from Genentech, or an alpha 4 integrin antibody such as natalizumab/ANTEGREN® available from Biogen, or others as noted above), drugs that treat symptoms secondary or related to ANCA-associated vasculitis (e.g., fungal and other infections) such as those noted herein, steroid such as corticosteroid (e.g., prednisolone, methylprednisolone such as SOLU-MEDROL™ methylprednisolone sodium succinate for injection, prednisone such as low-dose prednisone, dexamethasone, or glucocorticoid, e.g., via joint injection, including systemic corticosteroid therapy), non-lymphocyte-depleting immunosuppressive therapy (e.g., MMF or cyclosporine), cholesterol-lowering drug of the “statin” class (which includes cerivastatin (BAYCOL™), fluvastatin (LESCOL™), atorvastatin (LIPITOR™), lovastatin (MEVACOR™), pravastatin (PRAVACHOL™), and simvastatin (ZOCOR™)), estradiol, testosterone (optionally at elevated dosages; Stuve et al. Neurology 8:290-301 (2002)), androgen, hormone-replacement therapy, a TNF inhibitor such as an antibody to TNF-alpha, DMARD, NSAID, plasmapheresis or plasma exchange, trimethoprim-sulfamethoxazole (BACTRIM™, SEPTRA™), mycophenolate mofetil, H2-blockers or proton-pump inhibitors (during the use of potentially ulcerogenic immunosuppressive therapy), levothyroxine, cyclosporin A (e.g. SANDIMMUNE®), somatastatin analogue, cytokine, anti-cytokine antagonist or antibody, anti-metabolite, immunosuppressive agent, rehabilitative surgery, radioiodine, thyroidectomy, BAFF antagonist such as BAFF or BR3 antibodies or immunoadhesins, anti-CD40 receptor or anti-CD40 ligand (CD154), anti-1L-6 receptor antagonist/antibody, another B-cell surface antagonist or antibody such as a humanized 2H7 or other humanized or human CD20 antibody with rituximab, etc.

Preferred such medicaments are a chemotherapeutic agent, a cytotoxic agent, anti-integrin, gamma globulin, anti-CD4, cladribine, trimethoprimsulfamethoxazole, an H2-blocker, a proton-pump inhibitor, a corticosteroid, cyclosporine, cholesterol-lowering drug of the statin class, estradiol, testosterone, androgen, hormone-replacement drug, a TNF inhibitor, DMARD, NSAID (to treat, for example, musculoskeletal symptoms), levothyroxine, cyclosporin A, somatastatin analogue, cytokine antagonist or cytokine-receptor antagonist, anti-metabolite, BAFF antagonist such as BAFF antibody or BR3 antibody, especially a BAFF antibody, immunosuppressive agent, and another B-cell surface marker antibody, such as a combination of rituximab and a humanized 2H7 or other humanized CD20 antibody.

The more preferred such medicaments are a chemotherapeutic agent, an immunosuppressive agent, including an antibody against TNF-alpha, an antibody against CD40-CD40 ligand, and a BAFF antagonist such as a BAFF or BR3 antibody, a DMARD, a cytotoxic agent, an integrin antagonist, a NSAID, a cytokine antagonist, or a hormone, or a combination thereof. Immunosuppressants may be required, for example, for very active disease with major organ involvement, and include such agents as cyclophosphamide (CYTOXAN®), chlorambucil, leflunomide, MMF, azathioprine (IMURAN®), and methotrexate. BAFF antagonists may be useful in combination with the first medicament for efficacy.

Still more preferred are a steroid, chemotherapeutic agent, an immunosuppressive agent, a cytotoxic agent, an integrin antagonist, a cytokine antagonist, or a hormone, or a combination thereof, most preferably a steroid and/or an immunosuppressive agent, still most preferably, a corticosteroid and/or immunosuppressive agent.

In one particularly preferred embodiment, the second medicament is or comprises one or more steroids, for example, a corticosteroid, which is preferably prednisone, prednisolone, methylprednisolone, hydrocortisone, or dexamethasone. Such steroid is preferably administered in lower amounts than are used if the first medicament, e.g., CD20 antibody, is not administered to a patient treated with steroid. In a preferred aspect, the steroid is not administered with any second antibody exposure or is administered with the second exposure but in lower amounts than are used with the initial antibody exposure. Also preferred is wherein the steroid is not administered with third or later antibody exposures.

In a still further particularly preferred aspect, the second medicament is an immunosuppressive agent, more preferably cyclophosphamide, MMF, chlorambucil, azathioprine, leflunomide, or methotrexate, and preferably administered at least with the initial antibody exposure. In one embodiment, azathioprine, methotrexate, or MMF are preferably used instead of cyclophosphamide for the maintenance of remission.

In a yet further preferred aspect, the second medicament is a combination of one or more steroids and immunosuppressive agent.

Prophylactic treatment of the ANCA-associated vasculitis with fluconazole (DIFLUCAN™) orally for fungal infection may also be used, as well as trimethoprim-sulfamethoxazole (480 mg) three times weekly for prophylactic treatment of patients with pneumocystis carinii. Jayne and Rasmussen, supra.

All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as used hereinbefore or about from 1 to 99% of the heretofore-employed dosages. If such second medicaments are used at all, preferably, they are used in lower amounts than if the first medicament were not present, especially in subsequent dosings beyond the initial dosing with the first medicament, so as to eliminate or reduce side effects caused thereby.

For the re-treatment method herein, where a second medicament is administered in an effective amount with an antibody exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures. It is preferred that after the initial exposure, such as of steroid, the amount of such second medicament is reduced or eliminated so as to reduce the exposure of the subject to an agent with side effects such as prednisone, prednisolone, methylprednisolone, and cyclophosphamide.

As a specific example, treatment of patients with microscopic polyangiitis and Wegener's granulomatosis has three phases: (1) induction of remission, (2) maintenance of remission, and (3) treatment of relapse. Current induction therapy often consists of cyclophosphamide (CYTOXAN®) and corticosteroids. This includes a high dose of intravenous methylprednisolone for several days (e.g., one to five days), plus oral prednisone tapered over a period of time, such as 3-5 months. For aggressive disease, use of high-dose intravenous methylprednisolone for three days is recommended, combined with intravenous or oral cyclophosphamide. Tapering doses of prednisone preferably follows, along with cyclophosphamide maintenance for 12 to 18 months. When the first medicament is employed, such amount and frequency of dosing are preferably reduced further, since the lowest dosage of steroids that controls the disease should be used. Infection should be considered if the symptoms appear to exacerbate. For patients in sustained remission at 12 months, it is preferred that the use of all such second medicaments is discontinued at a faster rate with the first medicament herein administered than without it. Patients whose symptoms are under good control must, nevertheless, be closely followed at six-month intervals for signs and symptoms of relapse. During treatment with these agents, complete blood counts and liver function tests should be performed periodically.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

The antibody or antagonist herein is administered by any suitable means, including parenteral, topical, subcutaneous, intraperitoneal, intrapulmonary, intranasal, and/or intralesional administration. Parenteral infusions include intramuscular, intravenous (i.v.), intraarterial, intraperitoneal, or subcutaneous administration. Intrathecal administration is also contemplated (see, e.g., US 2002/0009444, Grillo-Lopez, A concerning intrathecal delivery of a CD20 antibody). In addition, the antibody or antagonist may suitably be administered by pulse infusion, e.g., with declining doses of the antibody or antagonist. Preferably, the dosing is given intravenously or subcutaneously, and more preferably by intravenous infusion(s).

If multiple exposures of antibody are provided, each exposure may be provided using the same or a different administration means. In one embodiment, each exposure is by intravenous administration. In another embodiment, each exposure is given by subcutaneous administration. In yet another embodiment, the exposures are given by both intravenous and subcutaneous administration.

In one embodiment, the CD20 antibody is administered as a slow intravenous infusion rather than an intravenous push or bolus. For example, a steroid such as prednisolone or methylprednisolone (e.g., about 80-120 mg i.v., more specifically about 100 mg i.v.) is administered about 30 minutes prior to any infusion of the CD20 antibody. The CD20 antibody is, for example, infused through a dedicated line.

For the initial dose of a multi-dose exposure to CD20 antibody, or for the single dose if the exposure involves only one dose, such infusion is preferably commenced at a rate of about 50 mg/hour. This may be escalated, e.g., at a rate of about 50 mg/hour increments every about 30 minutes to a maximum of about 400 mg/hour. However, if the subject is experiencing an infusion-related reaction, the infusion rate is preferably reduced, e.g., to half the current rate, e.g., from 100 mg/hour to 50 mg/hour. Preferably, the infusion of such dose of CD20 antibody (e.g., an about 1000-mg total dose) is completed at about 255 minutes (4 hours 15 min.). Optionally, the subjects receive a prophylactic treatment of acetaminophen/paracetamol (e.g., about 1 g) and diphenhydramine HCl (e.g., about 50 mg or equivalent dose of similar agent) by mouth about 30 to 60 minutes prior to the start of an infusion.

If more than one infusion (dose) of CD20 antibody is given to achieve the total exposure, the second or subsequent CD20 antibody infusions in this infusion embodiment are preferably commenced at a higher rate than the initial infusion, e.g., at about 100 mg/hour. This rate may be escalated, e.g., at a rate of about 100 mg/hour increments every about 30 minutes to a maximum of about 400 mg/hour. Subjects who experience an infusion-related reaction preferably have the infusion rate reduced to half that rate, e.g., from 100 mg/hour to 50 mg/hour. Preferably, the infusion of such second or subsequent dose of CD20 antibody (e.g., an about 1000-mg total dose) is completed by about 195 minutes (3 hours 15 minutes).

A discussion of methods of producing, modifying, and formulating such antibodies follows.

III. Production of Antibodies

The methods and articles of manufacture of the present invention use, or incorporate, an antibody that binds to a B-cell surface marker, especially one that binds to CD20. Accordingly, methods for generating such antibodies will be described here.

CD20 antigen to be used for production of, or screening for, antibody(ies) may be, e.g., a soluble form of CD20 or a portion thereof, containing the desired epitope. Alternatively, or additionally, cells expressing CD20 at their cell surface can be used to generate, or screen for, antibody(ies). Other forms of CD20 useful for generating antibodies will be apparent to those skilled in the art.

A description follows as to exemplary techniques for the production of the antibodies used in accordance with the present invention.

(i) Polyclonal Antibodies

Polyclonal antibodies are preferably raised in animals by multiple subcutaneous (s.c.) or intraperitoneal (i.p.) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C=NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In addition, antibodies comprising a variant Fc region with high affinity for FcγR are useful for treating diseases where an enhanced efficacy of effector cell function is desired, such as autoimmune diseases, as set forth, for example, in US 2005/0037000 and WO 2004/63351 (Macrogenics, Inc. STAVENHAGEN et al.).

(iii) Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson et al., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the CD20 antigen. Other such antibodies may bind CD20 and further bind a second B-cell surface marker. Alternatively, an anti-CD20 binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the B cell. Bispecific antibodies may also be used to localize cytotoxic agents to the B cell. These antibodies possess a CD20-binding arm and an arm that binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (V_{H}) connected to a light chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147: 60 (1991).

IV. Conjugates and Other Modifications of the Antibody

The antibody used in the methods or included in the articles of manufacture herein is optionally conjugated to a cytotoxic agent. For instance, the (CD20) antibody may be conjugated to a drug as described in WO2004/032828.

Chemotherapeutic agents useful in the generation of such antibody-cytotoxic agent conjugates have been described above.

Conjugates of an antibody and one or more small molecule toxins, such as a calicheamicin, a maytansine (U.S. Pat. No. 5,208,020), a trichothene, and CC1065 are also contemplated herein. In one embodiment of the invention, the antibody is conjugated to one or more maytansine molecules (e.g. about 1 to about 10 maytansine molecules per antibody molecule). Maytansine may, for example, be converted to May-SS-Me, which may be reduced to May-SH3 and reacted with modified antibody (Chari et al., Cancer Research 52: 127-131 (1992)) to generate a maytansinoid-antibody conjugate.

Alternatively, the antibody is conjugated to one or more calicheamicin molecules. The calicheamicin family of antibiotics is capable of producing double-stranded DNA breaks at sub-picomolar concentrations. Structural analogues of calicheamicin that may be used include, but are not limited to, γ₁^{I}, α₂^{I}, α₃^{I}, N-acetyl-γ₁^{I}, PSAG and θ^{I}₁ (Hinman et al., Cancer Research 53: 3336-3342 (1993) and Lode et al., Cancer Research 58: 2925-2928 (1998)).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates antibody conjugated with a compound with nucleolytic activity (e.g. a ribonuclease or a DNA endonuclease such as a deoxyribonuclease; DNase).

A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³² and radioactive isotopes of Lu.

Conjugates of the antibody and cytotoxic agent may be made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al., Cancer Research 52: 127-131 (1992)) may be used.

Alternatively, a fusion protein comprising the antibody and cytotoxic agent may be made, e.g. by recombinant techniques or peptide synthesis.

In yet another embodiment, the antibody may be conjugated to a “receptor” (such streptavidin) for utilization in tumor pretargeting wherein the antibody-receptor conjugate is administered to the subject, followed by removal of unbound conjugate from the circulation using a clearing agent and then administration of a “ligand” (e.g. avidin) that is conjugated to a cytotoxic agent (e.g. a radionucleotide).

The antibodies of the present invention may also be conjugated with a prodrug-activating enzyme that converts a prodrug (e.g. a peptidyl chemotherapeutic agent, see WO81/01145) to an active anti-cancer drug. See, for example, WO 88/07378 and U.S. Pat. No. 4,975,278.

The enzyme component of such conjugates includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes of this invention can be covalently bound to the antibody by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984)).

Other modifications of the antibody are contemplated herein. For example, the antibody may be linked to one of a variety of non-proteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are an especially preferred embodiment of the invention.

The antibodies disclosed herein may also be formulated as liposomes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and WO 97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al., J. National Cancer Inst. 81(19)1484 (1989).

Amino acid sequence modification(s) of protein or peptide antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells, Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of an enzyme, or a polypeptide that increases the serum half-life of the antibody.

Another type of variant is an amino acid substitution variant. These variants have at least one amino acid residue in the antibody molecule replaced by different residue. The sites of greatest interest for substitutional mutagenesis of antibodies include the hypervariable regions, but FR alterations are also contemplated. Conservative substitutions are shown in Table 3 under the heading of “preferred substitutions”. If such substitutions result in a change in biological activity, then more substantial changes, denominated “exemplary substitutions” in Table 3, or as further described below in reference to amino acid classes, may be introduced and the products screened.

TABLE 3

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (O)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. Such altering includes deleting one or more carbohydrate moieties found in the antibody, and/or adding one or more glycosylation sites that are not present in the antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec 13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered that has dual Fc regions and may thereby have enhanced complement lysis and ADCC capabilities. See Stevenson et al. Anti-Cancer Drug Design 3:219-230 (1989).

WO 00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region. Preferably the altered Fc region is a human IgG1 Fc region comprising or consisting of substitutions at one, two or three of these positions.

Antibodies with altered C1q binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.). The antibodies comprise an amino acid substitution at one or more of amino acid positions 270, 322, 326, 327, 329, 313, 333 and/or 334 of the Fc region thereof.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG3, or IgG₄) that is responsible for increasing the in vivo serum half-life of the IgG molecule. Antibodies with substitutions in an Fc region thereof and increased serum half-lives are also described in WO00/42072 (Presta, L.).

Engineered antibodies with three or more (preferably four) functional antigen binding sites are also contemplated (US Appln No. US2002/0004587 A1, Miller et al.).

V. Pharmaceutical Formulations

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary anti-CD20 antibody formulations are described in WO98/56418. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL polysorbate 80, and Sterile Water for Injection, pH 6.5.

Lyophilized formulations adapted for subcutaneous administration are described in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antibody are also contemplated. See, for example, US 2002/0136719A1 (Shenoy et al.).

The formulation herein may also contain more than one active compound (a second medicament as noted above) as necessary, preferably those with complementary activities that do not adversely affect each other. The type and effective amounts of such medicaments depend, for example, on the amount of antibody present in the formulation, and clinical parameters of the subjects. The preferred such medicaments are noted above.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

VI. Articles of Manufacture

In another embodiment of the invention, articles of manufacture containing materials useful for the treatment of ANCA-associated vasculitis described above are provided. In one aspect, the article of manufacture comprises (a) a container comprising an antagonist that binds to a B-cell surface marker (e.g., an antibody that so binds, including a CD20 antibody) (preferably the container comprises the antagonist or antibody and a pharmaceutically acceptable carrier or diluent within the container); and (b) a package insert with instructions for treating ANCA-associated vasculitis in a patient, wherein the instructions indicate that a dose of the antagonist or antibody of about 400 mg to 1.3 grams at a frequency of one to three doses is administered to the patient within a period of about one month.

Thus, the invention provides an article of manufacture comprising: a container comprising a CD20 antibody, or an antibody or antagonist that binds to a B-cell surface marker; and a package insert with instructions for treating ANCA-associated vasculitis in a patient, wherein the instructions indicate that a dose of the CD20 antibody, or the antibody or antagonist that binds to a B-cell surface marker, of about 400 mg to 1.3 grams, at a frequency of one to three doses, is administered to the patient within a period of about one month.

In a preferred embodiment, the article of manufacture herein further comprises a container comprising a second medicament, wherein the antagonist or antibody is a first medicament. This article further comprises instructions on the package insert for treating the patient with the second medicament, in an effective amount. The second medicament may be any of those set forth above, with an exemplary second medicament being a chemotherapeutic agent, an immunosuppressive agent, a cytotoxic agent, an integrin antagonist, a cytokine antagonist, or a hormone. The preferred second medicaments are those preferred as set forth above, and most preferred is a steroid or an immunosuppressive agent or both.

In another aspect, the invention provides an article of manufacture comprising: (a) a container comprising an antibody that binds to a B-cell surface marker (e.g., a CD20 antibody) (preferably the container comprises the antibody and a pharmaceutically acceptable carrier or diluent within the container); and (b) a package insert with instructions for treating ANCA-associated vasculitis in a subject, wherein the instructions indicate that an amount of the antibody is administered to the subject that is effective to provide an initial antibody exposure followed by a second antibody exposure, wherein the second exposure is not provided until from about 16 to 54 weeks from the initial exposure.

Preferably, such package insert is provided with instructions for treating ANCA-associated vasculitis in a subject, wherein the instructions indicate that an amount of the antibody is administered to the subject that is effective to provide an initial antibody exposure of about 0.5 to 4 grams followed by a second antibody exposure of about 0.5 to 4 grams, wherein the second exposure is not provided until from about 16 to 54 weeks from the initial exposure and each of the antibody exposures is provided to the subject as about one to four doses, preferably as a single dose or as two or three separate doses of antibody.

In a specific aspect, an article of manufacture is provided comprising:

(a) a container comprising an antibody that binds to a B-cell surface marker (e.g., a CD20 antibody) (preferably the container comprises the antibody and a pharmaceutically acceptable carrier or diluent within the container); and

(b) a package insert with instructions for treating ANCA-associated vasculitis in a subject, wherein the instructions indicate that an amount of the antibody is administered to the subject that is effective to provide an initial antibody exposure followed by a second antibody exposure, wherein the second exposure is not provided until from about 16 to 54 weeks from the initial exposure, and each of the antibody exposures is provided to the subject as a single dose or as two or three separate doses of antibody. Preferably, the antibody exposures are of about 0.5 to 4 grams.

In a preferred embodiment of these inventive aspects, the article of manufacture herein further comprises a container comprising a second medicament, wherein the antibody is a first medicament, and which article further comprises instructions on the package insert for treating the subject with the second medicament, in an effective amount. The second medicament may be any of those set forth above, with an exemplary second medicament being a chemotherapeutic agent, an immunosuppressive agent, a cytotoxic agent, an integrin antagonist, a cytokine antagonist, or a hormone, most preferably a steroid or an immunosuppressive agent, or both.

In all of these aspects, the package insert is on or associated with the container. Suitable containers include, for example, bottles, vials, syringes, etc. The containers may be formed from a variety of materials such as glass or plastic. The container holds or contains a composition that is effective for treating the ANCA-associated vasculitis and may have a sterile access port (for example the container may be an intravenous solution bag or a vial having a stopper pierceable by a hypodermic injection needle). At least one active agent in the composition is the antagonist or antibody. The label or package insert indicates that the composition is used for treating ANCA-associated vasculitis in a patient or subject eligible for treatment with specific guidance regarding dosing amounts and intervals of antagonist or antibody and any other medicament being provided. The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

Further details of the invention are illustrated by the following non-limiting Examples. The disclosures of all citations in the specification are expressly incorporated herein by reference.

### EXAMPLE 1

### Study of Efficacy of Rituximab in Patients with Wegener's Granulomatosis

This study assesses the superiority of efficacy and safety of rituximab (MABTHERA®/RITUXAN®) in a certain dosing regimen compared to placebo for treatment of signs and symptoms in patients with Wegener's granulomatosis exhibiting one or more symptoms of systemic disease.

Rituximab (1000 mg i.v.×2) is administered i.v. in two initial doses at days 1 and 15 with 1 mg/kg of oral prednisone daily (which is reduced to 40 mg/day at week 4, and tapered using a standardized tapering regimen resulting in complete discontinuation of prednisone over the following 3-5 months). This experimental regimen is compared to the same regimen except using rituximab placebo instead of rituximab, with 1:1 randomization between the two arms of the study, with about 48 patients per arm (total 96 patients). Active disease is defined as a Birmingham Vasculitis Activity Score/Wegener's granulomatosis (BVAS/WG) score of greater than 0. For trial inclusion, the patient's BVAS/WG score must be 3 or greater (or have been 3 or greater within 28 days of randomization). Each major item on the BVAS/WG evaluation form is scored 3 points. Each minor item is scored 1 point. In determining the degree of disease activity, the investigator will distinguish between active vasculitis (new/worse BVAS/WG items as opposed to persistent items) and permanent organ damage (caused by previously active vasculitis).

A severe flare is a new occurrence of one or more major BVAS/WG items. (Major items have a * on the BVAS/WG scoring sheet.) Generally, such flares are treated by increases in prednisone dose or cyclophosphamide dose.

Severe Wegener's granulomatosis occurs in a patient whose disease is not classifiable as limited, by definition below.

A limited flare is a new occurrence of one or more minor BVAS/WG items. Generally, such flares are treated with increases in prednisone dose or an increase in methotrexate dose.

Limited Wegener's granulomatosis occurs in a patient who meets the modified American College of Rheumatology (ACR) criteria for a diagnosis of Wegener's granulomatosis but who does not have disease that poses an immediate threat to either a critical individual organ or to the patient's life. Specifically, this means that:

The patient has no red blood cell casts in the urine.

If hematuria (but no +RBC casts) is present, the serum creatinine must be less than or equal to 1.4 and there must be evidence of no rise of creatinine more than 25% above the patient's baseline.

Pulmonary involvement must be circumscribed, such that the room air pO₂ is >70 mmHg or the room air O₂ saturation by pulse oximetry is >92%. Pulmonary hemorrhage may be treated as limited disease provided there is no evidence of progression of the process. In the absence of data on progression, pulmonary hemorrhage may be treated as severe disease at the discretion of the physician.

No disease may exist within any other critical organ (e.g., the gastrointestinal tract, eyes, central nervous system) that, without the immediate institution of maximal therapy (i.e., pulse methylprednisolone and daily oral cyclophosphamide), threatens the function of that organ and/or the patient's life.

A newly-diagnosed patient is a patient on his/her first treatment course of corticosteroids and/or a chemotherapeutic or immunosuppressive agent for Wegener's granulomatosis, with no history of increase in immunosuppressive therapy prior to entry into the study.

For the purpose of scoring BVAS/WG, persistent disease is defined as the presence of ongoing disease activity that was present at the previous trial evaluation (i.e., not new or worse activity).

A purified protein derivative skin test may be used to detect latent tuberculosis infection.

A refractory patient is a patient with a history of immunosuppressive therapy (corticosteroids and/or an immunosuppressive agent or chemotherapeutic agent) prior to the initiation of treatment for the Wegener's granulomatosis activity that makes the patient eligible for this study.

Patients will be considered to be in remission when their BVAS/WG score is 0.

This rituximab-based regimen challenges the current standard of care, and is intended to limit patient exposure to steroids and its known toxicities, and to demonstrate improved net clinical benefit. Patients are monitored for disease activity, use of additional immunosuppressants, steroid usage, and safety events over the trial length of one year, with the primary efficacy endpoint of the trial measured at 3 months, with follow-up to 16.8 months. Safety follow-up is required until 12 months following the last dose of rituximab or return of ANCA into the normal range, whichever occurs later.

The primary objective is to determine the proportion of patients achieving a BVAS/WG score of 0 and successful prednisone taper at 6 months, and no pre-specified adverse events.

It is predicted and expected that rituximab (or a humanized 2H7 substituted for rituximab) is effective in inducing remission (achieving BVAS/WG scores of 0) in at least 80% of the enrolled patients with Wegener's granulomatosis and to permit reduction in steroid doses over the control arm. BVAS/WG is expected to decrease from the score at entry to about 0.2 to 0.4 at week 14. C-reactive protein (mg/L) is expected to decrease from the level at entry to a range of about 3 to 11 by week 14. Mean prednisolone dose (mg/day) is expected to decrease from the value at entry to a statistically significant lower value at week 14. Relapse is expected to occur in fewer than five patients after a mean of 27 weeks. If the mean BVAS/WG at entry is 3.6, at 6 months of treatment, it is expected to decrease to a statistically significant value of 0.6. Intermittently active disease is expected to be observed in fewer than 70% of the patients. In contrast, the control arm is expected to show much less decrease in BVAS/WG and C-reactive protein, and in steroid use, and fewer patients in remission.

### EXAMPLE 2

### Study of Efficacy of Rituximab in Patients with Microscopic Polyangiitis

The protocol in Example 1 is followed except that the patients are treated for microscopic polyangiitis. It is expected that similar results will be observed as for Wegener's granulomatosis, i.e., that remission, as measured by the BVAS/WG score of 0, is expected to occur in at least 80% of the patients treated in the study arm and that steroid use is expected to decrease over the course of the study, which results are expected to be much better, in a statistically significant sense, than the control results.

### EXAMPLE 3

### Re-Treatment Study of Efficacy of Rituximab in Patients with Wegener's Granulomatosis

This study assesses the superiority of efficacy and safety of re-treatment with rituximab (MABTHERA®/RITUXAN®) compared to placebo in adult subjects with Wegener's granulomatosis. Study I examines acute disease, either first presentation or relapse (BVAS≧10; n=16); study II examines persistent disease (BVAS≧4; n=16). Patients receive rituximab (1 g i.v.) in three initial doses at days 1, 8, and 15 for Studies I and II. Concomitant therapy in Study I includes 1 mg/kg/day oral prednisone tapered according to the regimen in Example 1 and cyclophosphamide (according to standard treatment). Study II patients receive rituximab and 1 mg/kg/day oral prednisone tapered according to the regimen in Example 1. All subjects receive a second rituximab/placebo infusion course of 1000 mg i.v. separated by 14 days at weeks 24 and 26, respectively, without steroids or cyclophosphamide, whether the patients exhibited symptoms or were in complete remission. Courses of rituximab treatment must be separated by a minimum interval of 16 weeks.

The experimental regimens are compared to rituximab placebo+the same doses of tapered oral prednisone and cyclophosphamide (Study I), or tapered oral prednisone (Study II).

Changes in immunosuppressive drugs are not permitted during the studies, unless mandated by toxicity, and requests to taper a drug other than the oral prednisone must be discussed in advance with the Medical Monitor. Study personnel will be trained on how to properly administer rituximab. Subjects may be hospitalized for observation, particularly for their first infusion, at the discretion of the investigator. Rituximab must be administered under close supervision, and full resuscitation facilities must be immediately available.

Patients are monitored each month for 12 months for disease activity, use of additional immunosuppressants, flares of disease, prednisone usage, and safety events over the 52 weeks of the study. The primary efficacy endpoint of the trial is at 52 weeks, and efficacy measures are assessed by a unique Examining Assessor who is not involved with patient treatment or other study procedures. The patients are assessed for their BVAS/WG scores and successful prednisone taper. At the end of 52 weeks, subjects who received rituximab placebo or rituximab but demonstrate a BVAS/WG score of 0 and successful prednisone taper at 6 months will complete study participation. Subjects who received rituximab but have not demonstrated such score at 52 weeks are observed for 6 months following the last course of rituximab or until a BVAS/WG score of 0, whichever occurs first. Sites will be informed as to whether a subject must continue in follow-up, but not whether the subject received placebo or rituximab. Safety follow-up is required until 12 months following the last dose of rituximab or a BVAS/WG score of 0, whichever occurs later.

These rituximab-based regimens challenge the current standard of care, and are expected to demonstrate improved net clinical benefit, with the primary objective to determine the proportion of patients achieving the primary endpoint of a BVAS/WG score of 0, and successful prednisone taper.

It is predicted and expected that administration of rituximab or a humanized 2H7 to the subject in the protocols of Studies I and II set forth above will induce remission (achieving BVAS/WG scores of 0) in at least 80% of the enrolled patients with Wegener's granulomatosis and permit reduction in steroid doses over the control arm. BVAS/WG is expected to decrease from the score at entry to about 0.2 to 0.4 at week 14. C-reactive protein (mg/L) is expected to decrease from the level at entry to a range of about 3 to 11 by week 14. Mean steroid use for both Studies I and II is expected to decrease from the value at entry to a statistically significant lower value at week 14. Relapse is expected to occur in fewer than five patients after a mean of 27 weeks. These results are expected to be significantly better than those of the control arms for Studies I and II.

It is also expected that at about week 48-54, another 2-g dose of the rituximab given all at once or spread out over about 14-16 days in 1-gram amounts would be effective to treat Wegener's granulomatosis for the entire second year (causing a BVAS/WG score of 0 in at least 80% of the enrolled patients), with or without the steroid(s) and/or other immunosuppressive agents, with a marked improvement over control patients receiving rituximab placebo rather than rituximab. Thus, the rituximab (or a humanized 2H7) would be administered initially within about the 2-week time period, followed by another treatment at about 4-8 months, followed by another treatment at about one year from initial treatment (measured from the time any one of the doses was given), followed by treatment at about two years from initial treatment, with expected success, in about one-gram×2-4 dosing for each treatment, administered together, about weekly, or about every other week over about two to four weeks. This re-treatment protocol is expected to be successfully used for several years with little or no adverse effects.

### EXAMPLE 4

Second Re-treatment Study of Efficacy of Rituximab in Patients with Wegener's Granulomatosis

This study is the same as in Example 3 except that the initial dose of rituximab or rituximab placebo is given as 1000 mg i.v.×2 (on day 0, with the second infusion occurring on Day 15 +/−1 day), and the subsequent course of rituximab or placebo infusions administered at weeks 24 and 26 and consisting of 2 biweekly doses is administered only to those subjects in remission, e.g., those not exhibiting increasing disease activity, as by rising ANCA titers, sustained elevated ANCA titers, and other symptoms. All other criteria are the same.

It is predicted and expected that administration of rituximab or a humanized 2H7 to the subject in the scheduled redosing protocol set forth above will be effective in inducing remission (achieving BVAS/WG scores of 0) in at least 80% of the enrolled patients with Wegener's granulomatosis and in permitting reduction in steroid doses over the control arm. BVAS/WG is expected to decrease from the score at entry to about 0.2 to 0.4 at week 14. C-reactive protein (mg/L) is expected to decrease from the level at entry to a range of about 3 to 11 by week 14. Mean prednisolone dose (mg/day) is expected to decrease from the value at entry to a statistically significant lower value at week 14. Relapse is expected to occur in fewer than five patients after a mean of 27 weeks. These results are expected to be significantly better than those of the control.

It is also expected that at about week 48-54, another 2-g dose of the CD20 antibody (e.g., rituximab or a humanized 2H7) given all at once or spread out over about 14-16 days in 1-gram amounts would be effective to treat Wegener's granulomatosis for the entire second year (with at least 80% of the enrolled patients having a BVAS/WG score of 0), with or without the prednisone taper and i.v. methylprednisolone and/or other immunosuppressive agents. Thus, the CD20 antibody would be administered initially within about the 2-week time period, followed by another treatment at about 4-8 months, followed by another treatment at about one year from initial treatment (measured from the time any one of the doses was given), followed by treatment at about two years from initial treatment, with expected success, in about one-gram×2-4 dosing for each treatment, administered together, about weekly, or about every other week over about two to four weeks. The results of this treatment would be expected to be much better than those of the control with placebo. This re-treatment protocol is expected to be successfully used for several years with little or no adverse effects.

### EXAMPLE 5

### Third Re-treatment Study of Efficacy of Rituximab in Patients with Wegener's Granulomatosis

It is expected that Example 4 results would be successful if the patients were initially treated with rituximab and then re-treated with rituximab one year after first being treated, using the same dosing and other protocol of Example 4 except that rituximab is given at one-year intervals rather than six-month intervals.

### EXAMPLE 6

### Study of Efficacy of Rituximab in Subjects with Generalized ANCA-Associated Vasculitis

A randomized, multi-center, double-masked, placebo-controlled trial is performed in patients with generalized ANCA-associated vasculitis using rituximab. Two-hundred patients are randomized to either (1) conventional treatment (cyclophosphamide and corticosteroids, followed by azathioprine); or (2) rituximab (plus corticosteroids, initially) for remission induction, using 1 gram of rituximab on day 1 and again on day 15.

The primary clinical comparison is the ability of rituximab in this dosing regimen and corticosteroids to induce disease remissions, as measured by the cumulative disease activity at six months. Consistent with the standard duration of treatment for ANDA-associated vasculitis, patients in the conventional therapy arm will receive cyclophosphamide for up to 6 months followed by azathioprine, to complete a total length of treatment of 18 months. To assess the ability of rituximab to restore B-cell tolerance, patients in both arms of the trial will be followed for a total of 18 months.

It is expected that rituximab (or a humanized 2H7 substituted for rituximab) will induce stable remissions in the patients with ANCA-associated vasculitis and will re-establish B-cell tolerance to the ANCA target antigens in at least two thirds of the patients. It is also expected that rituximab or other CD20 antibody will be at least as effective as the conventional treatment regimen for induction and maintenance of disease remission, offering substantial advantages over standard therapy by virtue of its superior side-effect profile, e.g., much less toxic than chemotherapeutics and steroids, and better at restoring tolerance.

### EXAMPLE 7

### Re-treatment Study of Efficacy of Rituximab in Subjects with Severe Wegener's Granulomatosis or Severe Microscopic Polyangiitis

Twenty patients with active severe Wegener's granulomatosis or severe microscopic polyangiitis, positive ANCA test, and BVAS/WG score of at least 3, who are unresponsive to cyclophosphamide or have contraindications for cyclophosphamide use, are enrolled. See the definition of severe Wegener's granulomatosis in Example 1. The remission induction regimen consists of oral prednisone (1 mg/kg/day) and rituximab (1 gram at day 1 and 1 gram at day 15). By week 4, the prednisone is reduced to 40 mg/day. A standardized tapering regimen follows, resulting in complete discontinuation of prednisone over the following 16 weeks. This is compared with the same regimen, but with rituximab placebo rather than rituximab (control study). The protocol stipulates re-treatment with the same remission induction regimen at 6 months for all patients, whether they are experiencing a disease flare after reconstitution of B cells, whether they are asymptomatic with a recurrence of ANCA or ANCA titer rise coinciding or following reconstitution of B cells, or whether in complete remission. This re-treatment regimen includes the 1 g×2 two weeks apart for rituximab and rituximab placebo. A clinical flare in the absence of B cells is considered treatment failure. The patients are assessed monthly for one year.

It is expected that the patients in the treatment arm will tolerate rituximab infusions well and that their B cells will be depleted swiftly and all will achieve complete remission (BVAS/WG of 0) by three months. All patients in the treatment arm are expected to complete the glucocorticoid taper by 6 months. It is expected that after 12 months, no patient in the treatment arm will experience a clinical flare, and that B cells will return in most, if not all, such patients in the 12 months. Other than glucocorticoids, no additional immunosuppressive agents are expected to be necessary for induction of remission and maintenance of sustained remission (6 months or longer) in the rituximab-treated patients.

### EXAMPLE 8

### Humanized 2H7 Variants Useful Herein

Useful for purposes herein are humanized 2H7 antibodies comprising one, two, three, four, five, or six of the following CDR sequences:

CDR L1 sequence RASSSVSYXH wherein X is M or L (SEQ ID NO:35), for example, SEQ ID NO:4 (FIG. 1A),

CDR L2 sequence of SEQ ID NO:5 (FIG. 1A),

CDR L3 sequence QQWXFNPPT wherein X is S or A (SEQ ID NO:36), for example, SEQ ID NO:6 (FIG. 1A),

CDR H1 sequence of SEQ ID NO:10 (FIG. 1B),

CDR H2 sequence of AIYPGNGXTSYNQKFKG wherein X is D or A (SEQ ID NO:37), for example, SEQ ID NO:11 (FIG. 1B), and

CDR H3 sequence of VVYYSXXYWYFDV wherein the X at position 6 is N, A, Y, W, or D, and the X at position 7 is S or R (SEQ ID NO:38), for example, SEQ ID NO:12 (FIG. 1B).

The humanized 2H7 antibodies herein include those with heavy-chain amino acid sequences containing a C-terminal lysine and those without. The CDR sequences above are generally present within human variable light- and variable heavy-framework sequences, such as substantially the human consensus FR residues of human light-chain kappa subgroup I (V_{L}κI), and substantially the human consensus FR residues of human heavy-chain subgroup III (V_{H}III). See also WO 2004/056312 (Lowman et al.).

The variable heavy region may be joined to a human IgG chain constant region, wherein the region may be, for example, IgG1 or IgG3, including native-sequence and non-native-sequence constant regions.

In a preferred embodiment, such antibody comprises the variable heavy-domain sequence of SEQ ID NO:8 (v16, as shown in FIG. 1B), optionally also comprising the variable light-domain sequence of SEQ ID NO:2 (v16, as shown in FIG. 1A), which optionally comprises one or more amino acid substitution(s) at positions 56, 100, and/or 100a, e.g., D56A, N100A, or N100Y, and/or S100aR in the variable heavy domain and one or more amino acid substitution(s) at positions 32 and/or 92, e.g. M32L and/or S92A, in the variable light domain. Preferably, the antibody is an intact antibody comprising the light-chain amino acid sequence of SEQ ID NO:13 or 30, and heavy-chain amino acid sequence of SEQ ID NO:14, 15, 29, 31, 34, or 39, the sequence of SEQ ID NO:39 being given below.

A preferred humanized 2H7 antibody is ocrelizumab (Genentech, Inc.).

The antibody herein may further comprise at least one amino acid substitution in the Fc region that improves ADCC activity, such as one wherein the amino acid substitutions are at positions 298, 333, and 334, preferably S298A, E333A, and K334A, using Eu numbering of heavy-chain residues. See also U.S. Pat. No. 6,737,056, L. Presta.

Any of these antibodies may comprise at least one substitution in the Fc region that improves FcRn binding or serum half-life, for example, a substitution at heavy-chain position 434, such as N434W. See also U.S. Pat. No. 6,737,056, L. Presta.

Any of these antibodies may further comprise at least one amino acid substitution in the Fc region that increases CDC activity, for example, comprising at least a substitution at position 326, preferably K326A or K326W. See also U.S. Pat. No. 6,528,624, Idusogie et al.

Some preferred humanized 2H7 variants are those comprising the variable light domain of SEQ ID NO:2 and the variable heavy domain of SEQ ID NO:8, including those with or without substitutions in an Fc region (if present), and those comprising a variable heavy domain with alteration in SEQ ID NO:8 of N100A; or D56A and N100A; or D56A, N100Y, and S100aR; and a variable light domain with alteration in SEQ ID NO:2 of M32L; or S92A; or M32L and S92A.

M34 in the variable heavy domain of 2H7.v16 has been identified as a potential source of antibody stability and is another potential candidate for substitution.

In a summary of some various preferred embodiments of the invention, the variable region of variants based on 2H7.v16 comprise the amino acid sequences of v16 except at the positions of amino acid substitutions that are indicated in Table 4 below. Unless otherwise indicated, the 2H7 variants will have the same light chain as that of v16.

TABLE 4
Exemplary Humanized 2H7 Antibody Variants

| 2H7 | Heavy chain | Light chain | |
|---|---|---|---|
| Version | (V_{H}) changes | (V_{L}) changes | Fc changes |
| 16 for | | | — |
| reference | | | |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, |
| | | | E356D, M358L |
| 116 | D56A, N100A | M32L, S92A | S298A, K322A, K334A, |
| 138 | D56A, N100A | M32L, S92A | S298A, K326A, E333A, |
| | | | K334A, |
| 477 | D56A, N100A | M32L, S92A | S298A, K326A, E333A, |
| | | | K334A, N434W |
| 375 | — | — | K334L |
| 588 | — | — | S298A, K326A, E333A, |
| | | | K334A |
| 511 | D56A, N100Y, | M32L, S92A | S298A, K326A, E333A, |
| | S100aR | | K334A |

One preferred humanized 2H7 comprises 2H7.v16 variable light-domain sequence:

| | |
|---|---|
| DIQMTQSPSSLSASVGDRVTITCRASSSVSYMHWYQQKPGKAPKPLIYAPSNLASGVPSRFS | (SEQ ID NO: 2) |
| GSGSGTDFTLTISSLQPEDFATYYCQQWSFNPPTFGQGTKVEIKR; | |

and 2H7.v16 variable heavy-domain sequence:

| | |
|---|---|
| EVQLVESGGGLVQPGGSLRLSCAASGYTFTSYNMHWVRQAPGKGLEWVGAIYPGNGDTS | (SEQ ID NO: 8) |
| YNQKFKGRFTISVDKSKNTLYLQMNSLRAEDTAVYYCARVVYYSNSYWYFDVWGQGTL | |
| VTVSS. | |

Where the humanized 2H7.v16 antibody is an intact antibody, it may comprise the light-chain amino acid sequence:

| | |
|---|---|
| DIQMTQSPSSLSASVGDRVTITCRASSSVSYMHWYQQKPGKAPKPLIYAPSNLASGVPSRFS | (SEQ ID NO: 13) |
| GSGSGTDFTLTISSLQPEDFATYYCQQWSFNPPTFGQGTKVEIKRTVAAPSVFIFPPSDEQLK | |
| SGTASVVCLLNNFYPREAKVQWKVDNALQSGNSQESVTEQDSKDSTYSLSSTLTLSKADY | |
| EKHKVYACEVTHQGLSSPVTKSFNRGEC; | |

and the heavy-chain amino acid sequence of SEQ ID NO:14 or:

| | |
|---|---|
| EVQLVESGGGLVQPGGSLRLSCAASGYTFTSYNMHWVRQAPGKGLEWVGAIYPGNGDTS | (SEQ ID NO: 15) |
| YNQKFKGRFTISVDKSKNTLYLQMNSLRAEDTAVYYCARVVYYSNSYWYFDVWGQGTL | |
| VTVSSASTKGPSVFPLAPSSKSTSGGTAALGCLVKDYFPEPVTVSWNSGALTSGVHTFPAVL | |
| QSSGLYSLSSVVTVPSSSLGTQTYICNVNHKPSNTKVDKKVEPKSCDKTHTCPPCPAPELLG | |
| GPSVFLFPPKPKDTLMISRTPEVTCVVVDVSHEDPEVKFNWYVDGVEVHNAKTKPREEQY | |
| NSTYRVVSVLTVLHQDWLNGKEYKCKVSNKALPAPIEKTISKAKGQPREPQVYTLPPSREE | |
| MTKNQVSLTCLVKGFYPSDIAVEWESNGQPENNYKTTPPVLDSDGSFFLYSKLTVDKSRW | |
| QQGNVFSCSVMHEALHNHYTQKSLSLSPG. | |

Another preferred humanized 2H7 antibody comprises 2H7.v511 variable light-domain sequence:

| | |
|---|---|
| DIQMTQSPSSLSASVGDRVTITCRASSSVSYLHWYQQKPGKAPKPLIYAPSNLASGVPSRFS | (SEQ ID NO: 39) |
| GSGSGTDFTLTISSLQPEDFATYYCQQWAFNPPTFGQGTKVEIKR | |

and 2H7.v511 variable heavy-domain sequence:

| | |
|---|---|
| EVQLVESGGGLVQPGGSLRLSCAASGYTFTSYNMHWVRQAPGKGLEWVGAIYPGNGATS | (SEQ ID NO: 40) |
| YNQKFKGRFTISVDKSKNTLYLQMNSLRAEDTAVYYCARVVYYSYRYWYFDVWGQGTL | |
| VTVSS. | |

Where the humanized 2H7.v511 antibody is an intact antibody, it may comprise the light-chain amino acid sequence:

| | |
|---|---|
| DIQMTQSPSSLSASVGDRVTITCRASSSVSYLHWYQQKPGKAPKPLIYAPSNLASGVPSRFS | (SEQ ID NO: 30) |
| GSGSGTDFTLTISSLQPEDFATYYCQQWAFNPPTFGQGTKVEIKRTVAAPSVFIFPPSDEQLK | |
| SGTASVVCLLNNFYPREAKVQWKVDNALQSGNSQESVTEQDSKDSTYSLSSTLTLSKADY | |
| EKHKVYACEVTHQGLSSPVTKSFNRGEC | |

and the heavy-chain amino acid sequence of SEQ ID NO: 31 or:

| | |
|---|---|
| EVQLVESGGGLVQPGGSLRLSCAASGYTFTSYNMHWVRQAPGKGLEWVGAIYPGNGATS | (SEQ ID NO: 41) |
| YNQKFKGRFTISVDKSKNTLYLQMNSLRAEDTAVYYCARVVYYSYRYWYFDVWGQGTL | |
| VTVSSASTKGPSVFPLAPSSKSTSGGTAALGCLVKDYFPEPVTVSWNSGALTSGVHTFPAVL | |
| QSSGLYSLSSVVTVPSSSLGTQTYICNVNHKPSNTKVDKKVEPKSCDKTHTCPPCPAPELLG | |
| GPSVFLFPPKPKDTLMISRTPEVTCVVVDVSHEDPEVKFNWYVDGVEVHNAKTKPREEQY | |
| NATYRVVSVLTVLHQDWLNGKEYKCKVSNAALPAPIAATISKAKGQPREPQVYTLPPSREE | |
| MTKNQVSLTCLVKGFYPSDIAVEWESNGQPENNYKTTPPVLDSDGSFFLYSKLTVDKSRW | |
| QQGNVFSCSVMHEALHNHYTQKSLSLSPG. | |

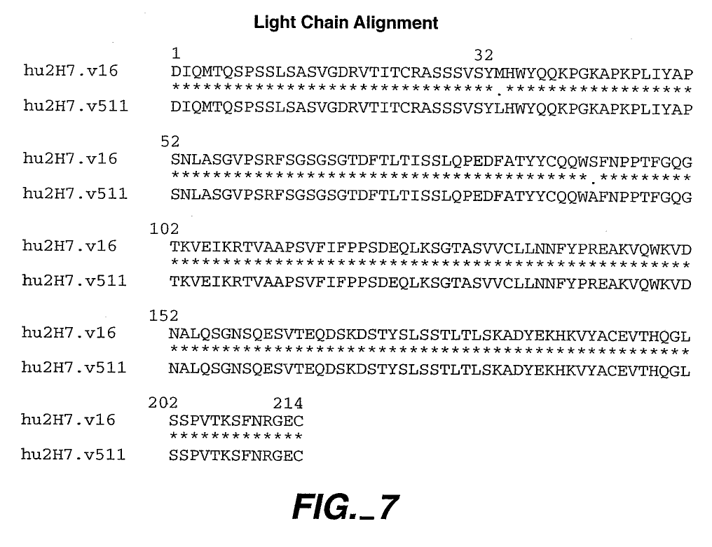

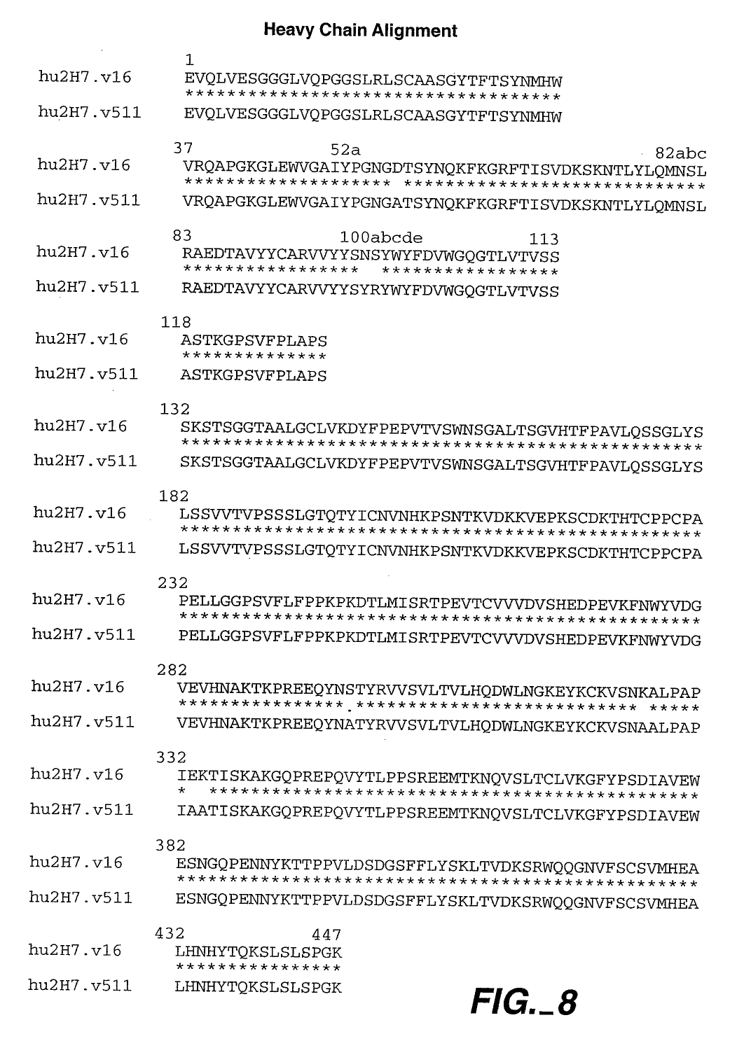

See FIGS. 7 and 8, which align the mature light and heavy chains, respectively, of humanized 2H7.v511 with humanized 2H7.v16 using the C-terminal lysine sequence for the heavy chain.

Where the humanized 2H7.v31 antibody is an intact antibody, it may comprise the light-chain amino acid sequence:

| | |
|---|---|
| DIQMTQSPSSLSASVGDRVTITCRASSSVSYLHWYQQKPGKAPKPLIYAPSNLASGVPSRFS | (SEQ ID NO: 13) |
| GSGSGTDFTLTISSLQPEDFATYYCQQWAFNPPTFGQGTKVEIKRTVAAPSVFIFPPSDEQLK | |
| SGTASVVCLLNNFYPREAKVQWKVDNALQSGNSQESVTEQDSKDSTYSLSSTLTLSKADY | |
| EKHKVYACEVTHQGLSSPVTKSFNRGEC | |

and the heavy-chain amino acid sequence of SEQ ID NO:15 or:

| | |
|---|---|
| EVQLVESGGGLVQPGGSLRLSCAASGYTFTSYNMHWVRQAPGKGLEWVGAIYPGNGDTS | (SEQ ID NO: 42) |
| YNQKFKGRFTISVDKSKNTLYLQMNSLRAEDTAVYYCARVVYYSNSYWYFDVWGQGTL | |
| VTVSSASTKGPSVFPLAPSSKSTSGGTAALGCLVKDYFPEPVTVSWNSGALTSGVHTFPAVL | |
| QSSGLYSLSSVVTVPSSSLGTQTYICNVNHKPSNTKVDKKVEPKSCDKTHTCPPCPAPELLG | |
| GPSVFLFPPKPKDTLMISRTPEVTCVVVDVSHEDPEVKFNWYVDGVEVHNAKTKPREEQY | |
| NATYRVVSVLTVLHQDWLNGKEYKCKVSNKALPAPIAATISKAKGQPREPQVYTLPPSREE | |
| MTKNQVSLTCLVKGFYPSDIAVEWESNGQPENNYKTTPPVLDSDGSFFLYSKLTVDKSRW | |
| QQGNVFSCSVMHEALHNHYTQKSLSLSPG | |
| or: | |
| EVQLVESGGGLVQPGGSLRLSCAASGYTFTSYNMHWVRQAPGKGLEWVGAIYPGNGATS | (SEQ ID NO: 43) |
| YNQKEKGRFTISVDKSKNTLYLQMNSLRAEDTAVYYCARVVYYSYRYWYFDVWGQGTL | |
| VTVSSASTKGPSVFPLAPSSKSTSGGTAALGCLVKDYFPEPVTVSWNSGALTSGVHTFPAVL | |
| QSSGLYSLSSVVTVPSSSLGTQTYICNVNHKPSNTKVDKKVEPKSCDKTHTCPPCPAPELLG | |
| GPSVFLFPPKPKDTLMISRTPEVTCVVVDVSHEDPEVKENWYVDGVEVHNAKTKPREEQY | |
| NATYRVVSVLTVLHQDWLNGKEYKCKVSNAALPAPIAATISKAKGQPREPQVYTLPPSREE | |
| MTKNQVSLTCLVKGFYPSDIAVEWESNGQPENNYKTTPPVLDSDGSFFLYSKLTVDKSRW | |
| QQGNVFSCSVMHEALHNHYTQKSLSLSPG. | |

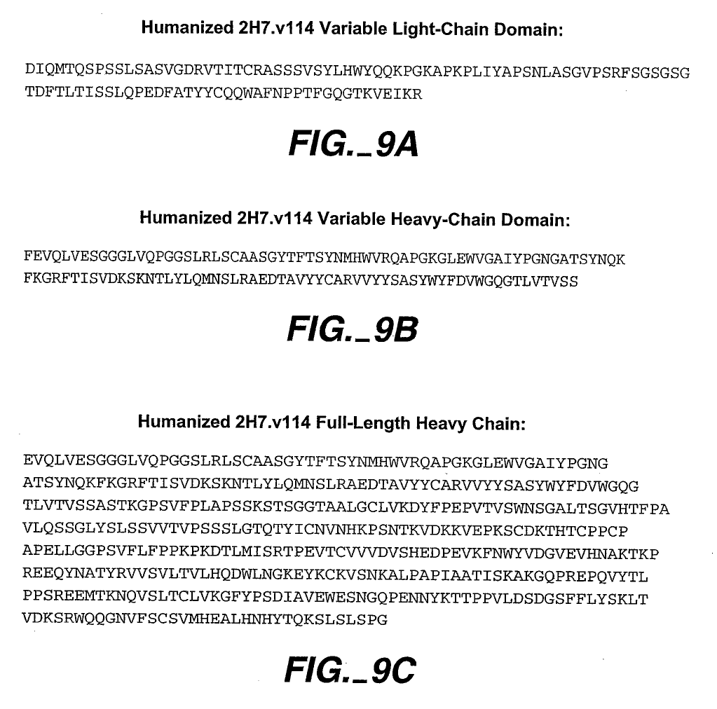

A preferred embodiment herein is where the antibody is humanized 2H7 comprising the variable domain sequences in SEQ ID NOS:2 and 8 (version 16). Another preferred embodiment herein is where the antibody is humanized 2H7 comprising the variable domain sequences in SEQ ID NOS:39 and 40 (version 511). Further preferred is where the antibody is humanized 2H7 comprising the variable domain sequences in SEQ ID NOS:32 and 33 (see FIG. 9 re version 114), such as one comprising the variable light-chain domain in SEQ ID NO:32 and the heavy-chain amino acid sequence of SEQ ID NO:34. Further preferred is wherein the antibody is humanized 2H7 comprising a variable heavy-chain domain with alteration N100A, or D56A and N100A, or D56A, N100Y, and S100aR in SEQ ID NO:8 and a variable light-chain domain with alteration M32L, or S92A, or M32L and S92A in SEQ ID NO:2.

What is claimed is:

1. A method of treating anti-neutrophil cytoplasmic antibodies-associated vasculitis (ANCA-associated vasculitis) in a patient comprising administering a CD20 antibody to the patient in a dose of about 400 mg to 1.3 grams at a frequency of one to three doses within a period of about one month.

2. The method of claim 1 wherein the dose is about 500 mg to 1.2 grams.

3. The method of claim 1 wherein the dose is about 750 mg to 1.1 grams.

4. The method of claim 1 wherein the antibody is administered in two to three doses.

5. The method of claim 1 wherein the antibody is administered in three doses.

6. The method of claim 1 wherein the antibody is administered within a period of about 2 to 3 weeks.

7. The method of claim 6 wherein the period is about three weeks.

8. The method of claim 1 wherein the ANCA-associated vasculitis is Wegener's granulomatosis.

9. The method of claim 1 wherein the ANCA-associated vasculitis is microscopic polyangiitis.

10. The method of claim 1 wherein a second medicament is administered in an effective amount, wherein the CD20 antibody is a first medicament.

11. The method of claim 10 wherein the second medicament is more than one medicament.

12. The method of claim 10 wherein the second medicament is a chemotherapeutic agent, an immunosuppressive agent, a disease-modifying anti-rheumatic drug (DMARD), a cytotoxic agent, an integrin antagonist, a non-steroidal anti-inflammatory drug (NSAID), a cytokine antagonist, or a hormone, or a combination thereof.

13. The method of claim 12 wherein the second medicament is a steroid or an immunosuppressive agent or both.

14. The method of claim 13 wherein the second medicament is a steroid.

15. The method of claim 14 wherein the steroid is a corticosteroid.

16. The method of claim 15 wherein the steroid is prednisone, prednisolone, methylprednisolone, hydrocortisone, or dexamethasone.

17. The method of claim 14 wherein the steroid is administered in lower amounts than are used if the CD20 antibody is not administered to a patient treated with steroid.

18. The method of claim 13 wherein the second medicament is an immunosuppressive agent.

19. The method of claim 18 wherein the immunosuppressive agent is cyclophosphamide, chlorambucil, mycophenolate mofetil, leflunomide, azathioprine, or methotrexate.

20. The method of claim 19 wherein the immunosuppressive agent is cyclophosphamide.

21. The method of claim 13 wherein the second medicament is a steroid and an immunosuppressive agent.

22. The method of claim 1 wherein the patient has never been previously treated with a CD20 antibody.

23. The method of claim 1 wherein the patient has not relapsed with the vasculitis.

24. The method of claim 1 wherein the antibody is a naked antibody.

25. The method of claim 1 wherein the antibody is conjugated with another molecule.

26. The method of claim 25 wherein the other molecule is a cytotoxic agent.

27. The method of claim 1 wherein the antibody is administered intravenously.

28. The method of claim 1 wherein the antibody is administered subcutaneously.

29. The method of claim 1 wherein no other medicament than the CD20 antibody is administered to the subject to treat the ANCA-associated vasculitis.

30. The method of claim 1 wherein the antibody is rituximab.

31. The method of claim 1 wherein the antibody is humanized 2H7 comprising the variable domain sequences in SEQ ID Nos. 2 and 8.

32. The method of claim 1 wherein the antibody is humanized 2H7 comprising the variable domain sequences in SEQ ID NOS:39 and 40.

33. The method of claim 1 wherein the antibody is humanized 2H7 comprising the variable domain sequences in SEQ ID NOS:32 and 33.

34. The method of claim 1 wherein the antibody is humanized 2H7 comprising a variable heavy-chain domain with alteration N100A, or D56A and N100A, or D56A, N100Y, and S100aR in SEQ ID NO:8 and a variable light-chain domain with alteration M32L, or S92A, or M32L and S92A in SEQ ID NO:2.

35. The method of claim 1 wherein the patient has a BVAS/WG score of 0 at six months after administration of the antibody.

36. The method of claim 1 wherein the patient has an elevated level of a anti-nuclear antibodies (ANA), anti-rheumatoid factor (RF) antibodies, creatinine, blood urea nitrogen, anti-endothelial antibodies, anti-neutrophil cytoplasmic antibodies (ANCA), or a combination thereof.

37. An article of manufacture comprising:
a. a container comprising a CD20 antibody; and
b. a package insert with instructions for treating anti-neutrophil cytoplasmic antibodies-associated vasculitis (ANCA-associated vasculitis) in a patient, wherein the instructions indicate that a dose of the CD20 antibody of about 400 mg to 1.3 grams at a frequency of one to three doses is administered to the patient within a period of about one month.

38. The article of claim 37 further comprising a container comprising a second medicament, wherein the CD20 antibody is a first medicament, further comprising instructions on the package insert for treating the patient with the second medicament.

39. The article of claim 38 wherein the second medicament is a chemotherapeutic agent, an immunosuppressive agent, a cytotoxic agent, an integrin antagonist, a cytokine antagonist, or a hormone.

40. The article of claim 38 wherein the second medicament is a steroid or an immunosuppressive agent or both.

41. A method of treating anti-neutrophil cytoplasmic antibodies-associated vasculitis (ANCA-associated vasculitis) in a subject comprising administering an effective amount of a CD20 antibody to the subject to provide an initial antibody exposure followed by a second antibody exposure, wherein the second exposure is not provided until from about 16 to 54 weeks from the initial exposure.

42. The method of claim 41 wherein the second exposure is not provided until from about 20 to 30 weeks from the initial exposure.

43. The method of claim 41 wherein the second exposure is not provided until from about 46 to 54 weeks from the initial exposure.

44. The method of claim 41 wherein each of the initial and second antibody exposures is provided in amounts of about 0.5 to 4 grams.

45. The method of claim 41 wherein each of the initial and second antibody exposures is provided in amounts of about 1.5 to 3.5 grams.

46. The method of claim 41 wherein each of the initial and second antibody exposures is provided in amounts of about 1.5 to 2.5 grams.

47. The method of claim 41 additionally comprising administering to the subject an effective amount of the CD20 antibody to provide a third antibody exposure, wherein the third exposure is not provided until from about 46 to 60 weeks from the initial exposure.

48. The method of claim 47 wherein the third antibody exposure is provided in an amount of about 0.5 to 4 grams.

49. The method of claim 47 wherein the third antibody exposure is provided in an amount of about 1.5 to 3.5 grams.

50. The method of claim 47 wherein the third antibody exposure is provided in an amount of about 1.5 to 2.5 grams.

51. The method of claim 47 wherein the third exposure is not provided until from about 46 to 55 weeks from the initial exposure.

52. The method of claim 47 wherein no further antibody exposure is provided until at least about 70-75 weeks from the initial exposure.

53. The method of claim 52 wherein no further antibody exposure is provided until about 74 to 80 weeks from the initial exposure.

54. The method of claim 41 wherein one or more of the antibody exposures is provided to the subject as a single dose of antibody.

55. The method of claim 54 wherein each antibody exposure is provided to the subject as a single dose of antibody.

56. The method of claim 41 wherein one or more of the antibody exposures is provided to the subject as separate doses of the antibody.

57. The method of claim 56 wherein each antibody exposure is provided as separate doses of the antibody.

58. The method of claim 56 wherein the separate doses are from about 2 to 3 doses.

59. The method of claim 56 wherein the separate doses constitute a first and second dose.

60. The method of claim 56 wherein the separate doses constitute a first, second, and third dose.

61. The method of claim 56 wherein a later dose is administered from about 1 to 20 days from the time the previous dose was administered.

62. The method of claim 56 wherein a later dose is administered from about 6 to 16 days from the time the previous dose was administered.

63. The method of claim 56 wherein a later dose is administered from about 14 to 16 days from the time the previous dose was administered.

64. The method of claim 56 wherein the separate doses are administered within a total period of between about 1 day and 4 weeks.

65. The method of claim 56 wherein the separate doses are administered within a total period of between about 1 and 25 days.

66. The method of claim 56 wherein the separate doses are administered about weekly, with the second dose being administered about one week from the first dose and any later dose being administered about one week from the previous dose.

67. The method of claim 56 wherein each separate dose of antibody is about 0.5 to 1.5 grams.

68. The method of claim 56 wherein each separate dose of antibody is about 0.75 to 1.3 grams.

69. The method of claim 41 wherein 4 to 20 antibody exposures are administered to the subject.

70. The method of claim 41 wherein a second medicament is administered in an effective amount with an antibody exposure, wherein the CD20 antibody is a first medicament.

71. The method of claim 70 wherein the second medicament is administered with the initial exposure.

72. The method of claim 70 wherein the second medicament is administered with the initial and second exposures.

73. The method of claim 70 wherein the second medicament is administered with all exposures.

74. The method of claim 70 wherein the second medicament is a chemotherapeutic agent, an immunosuppressive agent, a disease-modifying anti-rheumatic drug (DMARD), a cytotoxic agent, an integrin antagonist, a non-steroidal anti-inflammatory drug (NSAID), a cytokine antagonist, or a hormone, or a combination thereof.

75. The method of claim 70 wherein the second medicament comprises a steroid or an immunosuppressive agent or both.

76. The method of claim 75 wherein the second medicament is a steroid.

77. The method of claim 76 wherein the steroid is a corticosteroid.

78. The method of claim 77 wherein the steroid is prednisone, prednisolone, methylprednisolone, hydrocortisone, or dexamethasone.

79. The method of claim 76 wherein the steroid is administered in lower amounts than are used if the CD20 antibody is not administered to a subject treated with steroid.

80. The method of claim 75 wherein the second medicament is an immunosuppressive agent.

81. The method of claim 80 wherein the immunosuppressive agent is cyclophosphamide, chlorambucil, leflunomide, mycophenolate mofetil, azathioprine, or methotrexate.

82. The method of claim 81 wherein the immunosuppressive agent is cyclosphosphamide.

83. The method of claim 75 wherein the second medicament comprises a steroid and an immunosuppressive agent.

84. The method of claim 71 wherein the second medicament is not administered with the second exposure, or is administered in lower amounts than are used with the initial exposure.

85. The method of claim 41 wherein about 2-3 grams of the CD20 antibody is administered as the initial exposure.

86. The method of claim 85 wherein about 1 gram of the CD20 antibody is administered weekly for about three weeks as the initial exposure.

87. The method of claim 85 wherein the second exposure is at about six months from the initial exposure and is administered in an amount of about 2 grams.

88. The method of claim 85 wherein the second exposure is at about six months from the initial exposure and is administered as about 1 gram of the antibody followed in about two weeks by another about 1 gram of the antibody.

89. The method of claim 85 wherein about 1 gram of the CD20 antibody is administered followed in about two weeks by another about 1 gram of the antibody as the initial exposure.

90. The method of claim 89 wherein the second exposure is at about six months from the initial exposure and is administered in an amount of about 2 grams.

91. The method of claim 89 wherein the second exposure is at about six months from the initial exposure and is administered as about 1 gram of the antibody followed in about two weeks by another about 1 gram of the antibody.

92. The method of claim 85 wherein a steroid is administered to the subject before or with the initial exposure.

93. The method of claim 92 wherein the steroid is not administered with the second exposure or is administered with the second exposure but in lower amounts than are used with the initial exposure.

94. The method of claim 92 wherein the steroid is not administered with third or later exposures.

95. The method of claim 41 wherein the subject has never been previously treated with a CD20 antibody.

96. The method of claim 41 wherein the subject is in remission after the initial or a later antibody exposure.

97. The method of claim 41 wherein the subject is in remission when provided the second antibody exposure.

98. The method of claim 97 wherein the subject is in remission when provided all antibody exposures.

99. The method of claim 41 wherein the initial and second antibody exposures are with the same CD20 antibody.

100. The method of claim 41 wherein all antibody exposures are with the same CD20 antibody.

101. The method of claim 41 wherein the antibody is a naked antibody.

102. The method of claim 41 wherein the antibody is conjugated with another molecule.

103. The method of claim 102 wherein the other molecule is a cytotoxic agent.

104. The method of claim 41 wherein the antibody is administered intravenously.

105. The method of claim 104 wherein the antibody is administered intravenously for each antibody exposure.

106. The method of claim 41 wherein the antibody is administered subcutaneously.

107. The method of claim 106 wherein the antibody is administered subcutaneously for each antibody exposure.

108. The method of claim 41 wherein no other medicament than the CD20 antibody is administered to the subject to treat the ANCA-associated vasculitis.

109. The method of claim 41 wherein the ANCA-associated vasculitis is Wegener's granulomatosis.

110. The method of claim 41 wherein the ANCA-associated vasculitis is microscopic polyangiitis.

111. The method of claim 41 wherein the antibody is rituximab.

112. The method of claim 41 wherein the antibody is humanized 2H7 comprising the variable domain sequences in SEQ ID Nos. 2 and 8.

113. The method of claim 41 wherein the antibody is humanized 2H7 comprising the variable domain sequences in SEQ ID NOS:39 and 40.

114. The method of claim 41 wherein the antibody is humanized 2H7 comprising the variable domain sequences in SEQ ID NOS:32 and 33.

115. The method of claim 41 wherein the antibody is humanized 2H7 comprising a variable heavy-chain domain with alteration N100A, or D56A and N100A, or D56A, N100Y, and S100aR in SEQ ID NO:8 and a variable light-chain domain with alteration M32L, or S92A, or M32L and S92A in SEQ ID NO:2.

116. The method of claim 41 wherein the subject has a BVAS/WG score of 0 at six months after administration of the antibody.

117. The method of claim 41 wherein the subject has an elevated level of anti-nuclear antibodies (ANA), anti-rheumatoid factor (RF) antibodies, creatinine, blood urea nitrogen, anti-endothelial antibodies, anti-neutrophil cytoplasmic antibodies (ANCA), or a combination thereof.

118. The method of claim 41 wherein each of the antibody exposures is provided to the subject as a single dose or as two or three separate doses of antibody.

119. An article of manufacture comprising:
a. a container comprising a CD20 antibody; and
b. a package insert with instructions for treating anti-neutrophil cytoplasmic antibodies-associated vasculitis (ANCA-associated vasculitis) in a subject, wherein the instructions indicate that an amount of the antibody is administered to the subject that is effective to provide an initial antibody exposure followed by a second antibody exposure, wherein the second exposure is not provided until from about 16 to 54 weeks from the initial exposure.

120. The article of claim 119 wherein each of the antibody exposures is provided to the subject as a single dose or as two or three separate doses of antibody.

121. The article of claim 119 wherein each of the initial and second antibody exposures is provided in an amount of 0.5 to 4 grams.

122. The article of claim 119 further comprising a container comprising a second medicament, wherein the CD20 antibody is a first medicament, and further comprising instructions on the package insert for treating the subject with the second medicament.

123. The article of claim 122 wherein the second medicament is a chemotherapeutic agent, an immunosuppressive agent, a cytotoxic agent, an integrin antagonist, a cytokine antagonist, or a hormone.

124. The article of claim 125 wherein the second medicament is a steroid or an immunosuppressive agent or both.


SEQUENCE LISTING (43 sequences)

SEQ ID NO: 1 - protein, 107 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 2 - protein, 107 aa, Artificial sequence
  Sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 3 - protein, 108 aa, Homo sapiens
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 4 - protein, 26 aa, Artificial sequence
  Sequence is synthesized
RASSSVSYMHAPSNLASQQWSFNPPT

SEQ ID NO: 5 - protein, 26 aa, Artificial sequence
  Sequence is synthesized
RASSSVSYMHAPSNLASQQWSFNPPT

SEQ ID NO: 6 - protein, 27 aa, Artificial sequence
  Sequence is synthesized
RASQSISNYLAAASSLESQQYNSLPWT

SEQ ID NO: 7 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 8 - protein, 122 aa, Artificial sequence [sequence omitted]

SEQ ID NO: 9 - protein, 119 aa, Homo sapiens
EVQLVESGGGLVQPGGSLRLSCAASGFTFSSYAMSWVRQAPGKGLEWVAVISGDGGSTYYADSVKGRFTISRDNSKNTLTLQMNSLRAEDTAVYYCARGRVGYSLYDYWGQGTLVTVSS

SEQ ID NO: 10 - protein, 40 aa, Artificial sequence
  Sequence is synthesized
GYTFTSYNMHAIYPGNGDTSYNQKFKGVVYYSNSYWYFDV

SEQ ID NO: 11 - protein, 40 aa, Artificial sequence; identical to SEQ ID NO: 10
  Sequence is synthesized

SEQ ID NO: 12 - protein, 37 aa, Artificial sequence
  Sequence is synthesized
GFTFSSYAMSVISGDGGSTYYADSVKGGRVGYSLYDY

SEQ ID NO: 13 - protein, 213 aa, Artificial sequence
  Sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 452 aa, Artificial sequence
  Sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Artificial sequence
  Sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 17 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 18 - protein, 17 aa, Artificial sequence
  Xaa = Any Amino Acid except Cysteine
  unsure 3: unknown amino acid
  unsure 5: unknown amino acid
  unsure 7: unknown amino acid
  unsure 8: unknown amino acid
  unsure 9: unknown amino acid
  unsure 10: unknown amino acid
  unsure 12: unknown amino acid
  unsure 14: unknown amino acid
  unsure 15: unknown amino acid
  unsure 17: unknown amino acid
XCXDXLXXXXXXCXXXX

SEQ ID NO: 19 - protein, 17 aa, Artificial sequence
  Sequence is synthesized
ECFDLLVRAWVPCSVLK

SEQ ID NO: 20 - protein, 17 aa, Artificial sequence
  Sequence is synthesized
ECFDLLVRHWVPCGLLR

SEQ ID NO: 21 - protein, 17 aa, Artificial sequence
  Sequence is synthesized
ECFDLLVRRWVPCEMLG

SEQ ID NO: 22 - protein, 17 aa, Artificial sequence
  Sequence is synthesized
ECFDLLVRSWVPCHMLR

SEQ ID NO: 23 - protein, 17 aa, Artificial sequence [sequence omitted]

SEQ ID NO: 24 - protein, 16 aa, Artificial sequence
  Sequence is synthesized
QCFDRLNAWVPCSVLK

SEQ ID NO: 25 - protein, 17 aa, Artificial sequence
  Xaa = Any Amino Acid Except Cysteine
  unsure 3: unknown amino acid
  unsure 5: unknown amino acid
  unsure 8: unknown amino acid
  unsure 9: unknown amino acid
  unsure 14: unknown amino acid
  unsure 15: unknown amino acid
  unsure 17: unknown amino acid
XCXDXLVXXWVPCXXLX

SEQ ID NO: 26 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 27 - protein, 26 aa, Homo sapiens
TPCVPAECFDLLVRHCVACGLLRTPR

SEQ ID NO: 28 - protein, 213 aa, Artificial sequence
  Sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 452 aa, Artificial sequence
  Sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 213 aa, Artificial sequence; identical to SEQ ID NO: 28
  Sequence is synthesized

SEQ ID NO: 31 - protein, 451 aa, Artificial sequence
  Sequence is synthesized
EVQLVESGGGLVQPGGSLRLSCAASGYTFTSYNMHWVRQAPGKGLEWVGIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, Artificial sequence
  Sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 33 - protein, 123 aa, Artificial sequence
  Sequence is synthesized
FEVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 34 - protein, 451 aa, Artificial sequence
  Sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 10 aa, Artificial sequence
  Sequence is synthesized
  Unsure 9: X is M or L
RASSSVSYXH

SEQ ID NO: 36 - protein, 9 aa, Artificial sequence
  Sequence is synthesized
  Unsure 4: X is S or A
QQWXFNPPT

SEQ ID NO: 37 - protein, 17 aa, Artificial sequence
  Sequence is synthesized
  Unsure 8: X is D or A
AIYPGNGXTSYNQKFKG

SEQ ID NO: 38 - protein, 13 aa, Artificial sequence
  Sequence is synthesized
  Unsure 6: X  is N, A, Y, W, or D
  Unsure 7: X is S or R
VVYYSXXYWYFDV

SEQ ID NO: 39 - protein, 107 aa, Artificial sequence; identical to SEQ ID NO: 32
  Sequence is synthesized

SEQ ID NO: 40 - protein, 122 aa, Artificial sequence
  Sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 41 - protein, 451 aa, Artificial sequence
  Sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 451 aa, Artificial sequence
  Sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 451 aa, Artificial sequence; identical to SEQ ID NO: 41
  Sequence is synthesized